(19) United States
(12) Patent Application Publication
(10) Pub. No.: US 2024/0203453 A1
(43) Pub. Date: Jun. 20, 2024

(54) FLUORINE-CONTAINING ETHER COMPOUND, LUBRICANT FOR MAGNETIC RECORDING MEDIUM, AND MAGNETIC RECORDING MEDIUM

(71) Applicant: Resonac Corporation, Tokyo (JP)

(72) Inventors: Natsumi SHIBATA, Tokyo (JP); Naoya FUKUMOTO, Tokyo (JP); Ayano ASANO, Tokyo (JP); Tsuyoshi KATO, Tokyo (JP); Takuma KURODA, Tokyo (JP); Daisuke YAGYU, Tokyo (JP); Chizuru KASAHARA, Tokyo (JP); Yutaka TANJI, Tokyo (JP)

(73) Assignee: Resonac Corporation, Tokyo (JP)

(21) Appl. No.: 18/285,527

(22) PCT Filed: Apr. 6, 2022
(86) PCT No.: PCT/JP2022/017159

§ 371 Date: Oct. 4, 2023

(30) Foreign Application Priority Data
Apr. 8, 2021 (JP) 2021-065869

(51) Int. Cl. G11B 5/725; C08G 65/22; C10M 107/38; C10N 40/18; C10N 50/00

(52) U.S. Cl. CPC G11B 5/7257; C08G 65/226; C10M 107/38; C10M 2213/0606; C10N 2040/18; C10N 2050/025

(57) ABSTRACT

The present invention provides a fluorine-containing ether compound represented by the following formula.

R¹—CH₂—R²—CH₂—R³—CH₂—R⁴—CH₂—R⁵—CH₂—R⁶—CH₂—R⁷

(In Formula (1), R², R⁴, and R⁶ are perfluoropolyether chains, R³ is Formula (2), R⁵ is Formula (3), and R¹ and R⁷ are each independently a terminal group having two or three polar groups, in which individual polar groups are bound to different carbon atoms and the carbon atoms to which the polar groups are bound are bound to each other via a linking group containing a carbon atom to which no polar group is bound).

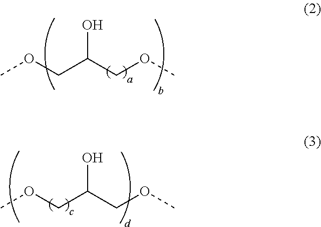

### TECHNICAL FIELD

The present invention relates to a fluorine-containing ether compound, a lubricant for a magnetic recording medium, and a magnetic recording medium.

Priority is claimed on Japanese Patent Application No. 2021-065869, filed Apr. 8, 2021, the content of which is incorporated herein by reference.

### BACKGROUND ART

Development of magnetic recording media suitable for high recording densities is underway to improve the recording densities of magnetic recording/reproducing devices.

As a conventional magnetic recording medium, there is a magnetic recording medium in which a recording layer is formed on a substrate and a protective layer made of carbon or the like is formed on the recording layer. The protective layer protects information recorded in the recording layer and enhances the slidability of a magnetic head. In addition, the protective layer covers the recording layer to prevent metal contained in the recording layer from being corroded by environmental substances.

However, sufficient durability of the magnetic recording medium cannot be obtained by simply providing the protective layer on the recording layer. Therefore, a lubricant is applied to the surface of the protective layer to form a lubricating layer with a thickness of about 0.5 to 3 nm. The lubricating layer improves the durability and protective power of the protective layer and prevents contamination substances from intruding into the magnetic recording medium.

After forming the lubricating layer on the surface of the protective layer, a burnishing step may be performed to remove projections and particles present on the surface of the magnetic recording medium and improve the smoothness of the surface.

As a lubricant that is used at the time of forming a lubricating layer in a magnetic recording medium, there is, for example, a lubricant containing a fluorine-based polymer having a repeating structure containing —CF₂— and having a polar group such as a hydroxyl group at a terminal.

For example, Patent Document 1 discloses a magnetic disk provided with a lubricating layer containing a fluorine-containing ether compound containing three perfluoropolyether chains in its molecule and having the same structure at both terminals.

Patent Document 2 discloses a magnetic disk provided with a lubricating layer containing a fluorine-containing ether compound containing three perfluoropolyether chains in its molecule and having two different terminal structures.

In addition, Patent Document 3 discloses a magnetic disk having a lubricating layer containing a lubricant containing three perfluoropolyether chains in its molecule and having two hydroxyl groups in linking groups provided between the perfluoropolyether chains.

### CITATION LIST

### Patent Document

### Patent Document 1

PCT International Publication No. WO2018/116742

### Patent Document 2

PCT International Publication No. WO2017/145995

### Patent Document 3

United States Patent Application, Publication No. 2016/0260452

### SUMMARY OF INVENTION

### Technical Problem

There is a demand for a further decrease in the flying height of a magnetic head in magnetic recording/reproducing devices. This requires a further decrease in the thickness of lubricating layers in magnetic recording media.

However, if the thickness of lubricating layers is reduced, the corrosion resistance of magnetic recording media may become insufficient. In particular, in a case where a tape burnishing process is performed on the surface of a magnetic recording medium after forming a lubricating layer, the corrosion resistance of the magnetic recording medium is likely to be insufficient. For this reason, there is a demand for a lubricating layer which is highly effective in suppressing corrosion of magnetic recording media.

The present invention has been made in consideration of the above circumstances, and an object of the invention is to provide a suitable fluorine-containing ether compound as a material for a lubricant for a magnetic recording medium with which a lubricating layer highly effective in suppressing corrosion of a magnetic recording medium can be formed.

In addition, another object of the present invention is to provide a lubricant for a magnetic recording medium which contains the fluorine-containing ether compound of the present invention and with which a lubricating layer highly effective in suppressing corrosion of a magnetic recording medium can be formed.

In addition, still another object of the present invention is to provide a magnetic recording medium in which a lubricating layer containing the fluorine-containing ether compound of the present invention is provided and which has an excellent corrosion resistance.

### Solution to Problem

That is, the present invention relates to the following features.

A first aspect of the present invention provides the following fluorine-containing ether compound.

[1] A fluorine-containing ether compound represented by Formula (1) below.

R¹—CH₂—R²—CH₂—R³—CH₂—R⁴—CH₂—R⁵—CH₂—R⁶—CH₂—R⁷ (1)

(In Formula (1), R², R⁴, and R⁶ are the same or different perfluoropolyether chains, R³ is a linking group represented by Formula (2) below, R⁵ is a linking group represented by Formula (3) below, and R¹ and R⁷ are each independently a terminal group having two or three polar groups, in which individual polar groups are bound to different carbon atoms and the carbon atoms to which the polar groups are bound are bound to each other via a linking group containing a carbon atom to which no polar group is bound.)

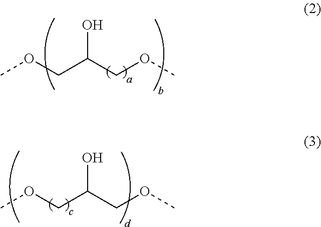

(In Formula (2), a is an integer of 1 to 3 and b is an integer of 1 to 2, and in Formula (3), c is an integer of 1 to 3 and d is an integer of 1 to 2, provided that a in Formula (2) and c in Formula (3) are not 1 at the same time.)

The fluorine-containing ether compound of the first aspect of the present invention preferably includes features described in [2] to [8] below. It is also preferable to arbitrarily combine two or more of the features described in [2] to [8] below.

[2] The fluorine-containing ether compound according to [1], in which a in Formula (2) above is an integer of 2 to 3, and c in Formula (3) above is an integer of 2 to 3.

[3] The fluorine-containing ether compound according to [1] or [2], in which all of the polar groups in R¹ and R⁷ in Formula (1) above are hydroxyl groups.

[4] The fluorine-containing ether compound according to any one of [1] to [3], in which —R¹ and —R⁷ in Formula (1) above are each independently a terminal group represented by Formulae (4-1) to (4-5) below.

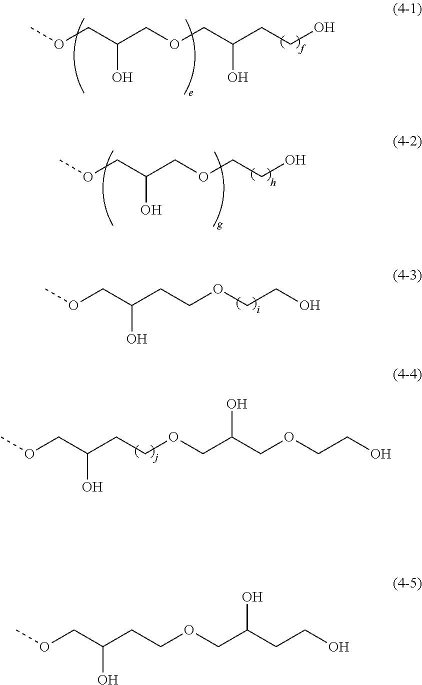

(In Formula (4-1), e is an integer of 0 to 1 and f is an integer of 1 to 4.)

(In Formula (4-2), g is an integer of 1 to 2 and h is an integer of 1 to 3.)

(In Formula (4-3), i is an integer of 1 to 3.)

(In Formula (4-4), j is an integer of 1 to 2.)

[5] The fluorine-containing ether compound according to any one of [1] to [4], in which R², R⁴, and R⁶ in Formula (1) above are each independently any of Formulae (5) to (9) below.

—CF₂O—(CF₂CF₂O)ₖ—(CF₂O)ₗ—CF₂— (5)

(In Formula (5), k and l indicate the average degree of polymerization and each independently represent 0.1 to 20.)

—CF₂O—(CF₂CF₂O)ₘ—CF₂— (6)

(In Formula (6), m indicates the average degree of polymerization and represents 0.1 to 20.)

—CF₂CF₂O—(CF₂CF₂CF₂O),—CF₂CF₂— (7)

(In Formula (7), n indicates the average degree of polymerization and represents 0.1 to 20.)

—CF₂CF₂CF₂O—(CF₂CF₂CF₂CF₂O)ₒ—CF₂CF₂CF₂— (8)

(In Formula (8), o indicates the average degree of polymerization and represents 0.1 to 10.)

—CF(CF₃)O—(CF₂CF(CF₃)O)ₚ—CF(CF₃)— (9)

(In Formula (9), p indicates the average degree of polymerization and represents 0.1 to 20.)

[6] The fluorine-containing ether compound according to any one of [1] to [5], in which R¹ and R⁷ in Formula (1) above are the same as each other.

[7] The fluorine-containing ether compound according to any one of [1] to [6], in which R² and R⁶ in Formula (1) above are the same as each other.

[8] The fluorine-containing ether compound according to any one of [1] to [7], in which a number average molecular weight thereof is within a range of 500 to 10,000.

A second aspect of the present invention is to provide a lubricant for a magnetic recording medium below.

[9] A lubricant for a magnetic recording medium including: the fluorine-containing ether compound according to any one of [1] to [8].

A third aspect of the present invention is to provide a magnetic recording medium below.

[10] A magnetic recording medium, in which at least a magnetic layer, a protective layer, and a lubricating layer are sequentially provided on a substrate, and the lubricating layer contains the fluorine-containing ether compound according to any one of [1] to [8].

The magnetic recording medium of the third aspect of the present invention preferably includes features described in below.

[11] The magnetic recording medium according to [10], in which the average film thickness of the lubricating layer is 0.5 nm to 2.0 nm.

### Advantageous Effects of Invention

The fluorine-containing ether compound of the present invention is the compound represented by Formula (1) above, and therefore can be used as a material for a lubricant for a magnetic recording medium with which a lubricating layer highly effective in suppressing corrosion of a magnetic recording medium can be formed.

Since the lubricant for a magnetic recording medium of the present invention contains the fluorine-containing ether compound of the present invention, a lubricating layer highly effective in suppressing corrosion of a magnetic recording medium can be formed.

The magnetic recording medium of the present invention is provided with the lubricating layer containing the fluorine-containing ether compound of the present invention, and therefore has an excellent corrosion resistance. For this reason, the magnetic recording medium of the present invention has excellent reliability and durability. In addition, since the magnetic recording medium of the present invention is provided with the lubricating layer highly effective in suppressing corrosion of the magnetic recording medium, the thickness of a protective layer and/or the lubricating layer can be reduced.

### BRIEF DESCRIPTION OF DRAWING

FIG. 1 is a schematic cross-sectional view showing an example of one embodiment of a magnetic recording medium of the present invention.

### DESCRIPTION OF EMBODIMENT

The present inventors have conducted extensive studies focusing on a chain skeleton and terminal groups of a fluorine-containing ether compound to solve the above-described problems.

As a result, they have found that a fluorine-containing ether compound may be obtained which has a chain skeleton, in which three perfluoropolyether chains are bound via two specific linking groups in which one or more hydroxyl groups are bound to a chain structure including methylene groups (—CH₂—) and ether bonds (—O—), and in which specific terminal groups having two or three polar groups are bound to both ends of the chain skeleton via methylene groups.

In the fluorine-containing ether compound having the above-described chain skeleton, one or both of the two linking groups have a methylene chain with 2 or 3 carbon atoms between the central perfluoropolyether chain and a carbon atom to which a hydroxyl group is bound. As a result, it is inferred that a lubricating layer containing the fluorine-containing ether compound having the above-described chain skeleton would have appropriate hydrophobicity.

In addition, when the lubricating layer containing the fluorine-containing ether compound having the above-described chain skeleton is formed on a protective layer, both ends of the perfluoropolyether chain placed at the center of the chain skeleton adhere closely to the protective layer due to hydroxyl groups in the linking groups arranged between perfluoropolyether chains. For this reason, a lubricant containing the fluorine-containing ether compound having the above-described chain skeleton is likely to wet and spread on the protective layer, can uniformly adhere closely to the protective layer, and can form a lubricating layer with a high coating rate and favorable adhesion properties compared to a case where a lubricant contains a fluorine-containing ether compound having one or two perfluoropolyether chains and having the same number of carbon atoms as the above-described chain skeleton.

Moreover, at both ends of the above-described chain skeleton, terminal groups each independently having two or three polar groups are arranged, in which individual polar groups are bound to different carbon atoms and the carbon atoms to which the polar groups are bound are bound to each other via a linking group containing a carbon atom to which no polar group is bound. The two or three polar groups in these terminal groups can be oriented so as to adhere closely to the protective layer, unlike the terminal groups in which carbon atoms to which polar groups are bound are bound to each other. For this reason, for example, it is possible to form a lubricating layer with a higher coating rate and more favorable adhesion properties than a lubricating layer containing a fluorine-containing ether compound having terminal groups in which carbon atoms to which polar groups are bound are bound to each other. Furthermore, since the terminal groups have a linking group containing a carbon atom to which no polar group is bound, the hydrophobicity becomes more favorable than that of the lubricating layer containing a fluorine-containing ether compound having terminal groups in which carbon atoms to which polar groups are bound are bound to each other.

In this manner, the lubricating layer containing the fluorine-containing ether compound in which the above-described specific terminal groups are bound to both ends of the above-described chain skeleton has appropriate hydrophobicity and favorable adhesion properties with respect to the protective layer. For this reason, it is inferred that the above-described lubricating layer would be highly effective in suppressing corrosion of a magnetic recording medium.

The present inventors have conducted further studies to confirm that the lubricating layer containing the above-described fluorine-containing ether compound is formed on the protective layer and is highly effective in suppressing corrosion of a magnetic recording medium, thus leading to realization of the present invention.

Hereinafter, preferable examples of a fluorine-containing ether compound, a lubricant for a magnetic recording medium (hereinafter, abbreviated as a “lubricant” in some cases), and a magnetic recording medium of the present invention will be described in detail. The present invention is not limited to only the embodiment shown below.

Numbers, amounts, positions, ratios, materials, configurations, and the like in the present invention can be added, omitted, replaced, or modified within a scope not departing from the gist of the present invention.

### [Fluorine-Containing Ether Compound]

A fluorine-containing ether compound of the present embodiment is represented by Formula (1) below.

R¹—CH₂—R²—CH₂—R³—CH₂—R⁴—CH₂—R⁵—CH₂—R⁶—CH₂—R⁷ (1)

(In Formula (1), R², R⁴, and R⁶ are the same or different perfluoropolyether chains, R³ is a linking group represented by Formula (2) below, R⁵ is a linking group represented by Formula (3) below, and R¹ and R⁷ are each independently a terminal group having two or three polar groups, in which individual polar groups are bound to different carbon atoms and the carbon atoms to which the polar groups are bound are bound to each other via a linking group containing a carbon atom to which no polar group is bound.)

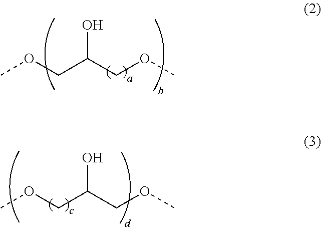

(In Formula (2), a is an integer of 1 to 3 and b is an integer of 1 to 2, and in Formula (3), c is an integer of 1 to 3 and d is an integer of 1 to 2, provided that a in Formula (2) and c in Formula (3) are not 1 at the same time.)

(R¹ and R⁷)

R¹ and R⁷ in the fluorine-containing ether compound represented by Formula (1) above are terminal groups each independently containing two or three polar groups, in which individual polar groups are bound to different carbon atoms and the carbon atoms to which the polar groups are bound are bound to each other via a linking group containing a carbon atom to which no polar group is bound.

In the fluorine-containing ether compound represented by Formula (1), R¹ and R⁷ each independently have two or three polar groups. For this reason, in a case where a lubricating layer is formed on a protective layer using a lubricant containing this fluorine-containing ether compound, a suitable interaction is generated between the lubricating layer and the protective layer.

Examples of polar groups include a hydroxyl group (—OH), an amino group (—NH₂), a carboxy group (—COOH), an aldehyde group (—COH), a carbonyl group (—CO—), and a sulfo group (—SO₃H). It is preferable that R¹ and/or R⁷ contain a hydroxyl group as a polar group, and it is more preferable that all of polar groups in R¹ and R⁷ be hydroxyl groups. In a case of a fluorine-containing ether compound in which R¹ and/or R⁷ have a hydroxyl group, when a lubricating layer is formed on a protective layer using a lubricant containing this fluorine-containing ether compound, a more suitable interaction is generated between the lubricating layer and the protective layer.

In the fluorine-containing ether compound represented by Formula (1), the total number of polar groups in R¹ and polar groups in R⁷ is 4 to 6. Since the above-described total number is 4 or more, the lubricating layer containing the fluorine-containing ether compound has high adhesiveness (adhesion properties) with respect to the protective layer. In addition, since the above-described total number is 6 or less, it is possible to prevent pickup which is adhesion of the fluorine-containing ether compound to a magnetic head as foreign matter (smears) due to excessively high polarity in a magnetic recording medium having the lubricating layer containing the fluorine-containing ether compound.

The number of polar groups in R¹ is preferably the same as the number of polar groups in R⁷. That is, it is preferable that R¹ and R⁷ each have two polar groups or each have three polar groups. In this case, since the lubricant containing the fluorine-containing ether compound adheres closely to the protective layer with a good balance, a lubricating layer which has a high coating rate and is highly effective in suppressing corrosion of a magnetic recording medium is likely to be obtained. In particular, in a case where R¹ and R⁷ each independently contain three polar groups, the polar groups contained in the fluorine-containing ether compound firmly adhere closely to the protective layer, and therefore the coating rate increases and the effect of suppressing corrosion of a magnetic recording medium increases.

Each polar group in R¹ and R⁷ is bound to a different carbon atom. In R¹ and R⁷, the carbon atoms to which the polar groups are bound are bound to each other via a linking group containing a carbon atom to which no polar group is bound. For this reason, the fluorine-containing ether compound represented by Formula (1) has favorable hydrophobicity compared to, for example, a compound having terminal groups in which carbon atoms to which polar groups are bound are bound to each other. As a result, it is inferred that the lubricating layer containing the fluorine-containing ether compound represented by Formula (1) can prevent water from intruding and effectively suppress corrosion of a magnetic recording medium.

In addition, in a case where the carbon atoms to which the polar groups are bound are bound to each other via a linking group containing a carbon atom to which no polar group is bound, the linking group containing a carbon atom to which no polar group is bound allows both the most terminal polar group (terminal polar group) and the polar group adjacent to the terminal polar group to be oriented so as to adhere closely to the protective layer. From this, it is inferred that, in the case where the carbon atoms to which the polar groups are bound are bound to each other via a linking group containing a carbon atom to which no polar group is bound, a lubricating layer highly effective in suppressing corrosion of a magnetic recording medium would be obtained.

On the other hand, in a case where the fluorine-containing ether compound contained in the lubricating layer has terminal groups in which a carbon atom to which the most terminal polar group (terminal polar group) is bound is bound to a carbon atom to which a polar group adjacent to the terminal polar group is bound, either the terminal polar group or the polar group adjacent to the terminal polar group is oriented in the opposite direction with respect to the protective layer. For this reason, either the terminal polar group or the polar group adjacent to the terminal polar group is less likely to adhere closely to the protective layer, resulting in poor adhesion properties between the lubricating layer and the protective layer.

In R¹ and R⁷, in a case where a linking group between a carbon atom to which a terminal polar group is bound and a carbon atom to which a polar group adjacent to the terminal polar group is bound contains an oxygen atom (for example, in cases of terminal groups represented by Formulae (4-2), (4-3), and (4-4) below), the above-described linking group in R¹ and R⁷ preferably has a linear structure containing 2 to 5 carbon atoms to which no polar group is bound. Even in the case where the above-described linking group contains an oxygen atom, if the linking group has a linear structure containing two or more carbon atoms to which no polar group is bound, a fluorine-containing ether compound having favorable hydrophobicity is obtained. In addition, if the above-described linking group has a linear structure containing 5 or less carbon atoms, problems are not caused in adhesion properties with respect to the protective layer due to a too hydrophobic linking group. In particular, in a case where R¹ and R⁷ each independently have three polar groups, a lubricating layer having superior adhesion properties with respect to a protective layer can be formed. Accordingly, a lubricating layer containing a fluorine-containing ether compound in which the above-described linking group in R¹ and R⁷ have a linear structure containing the number of carbon atoms within the above-described range can have excellent adhesion properties with respect to a protective layer and can prevent water from intruding due to appropriate hydrophobicity, thereby obtaining a high effect of suppressing corrosion of a magnetic recording medium.

In addition, in the case where the above-described linking group in R¹ and R⁷ contains an oxygen atom, the above-described linking group preferably has a linear structure consisting of 3 to 9 atoms and more preferably a linear structure consisting of 3 to 6 atoms. When the number of atoms in the above-described linking group is within the above-described range, the molecular mobility is appropriate and intramolecular aggregation is less likely to occur. For this reason, a fluorine-containing ether compound with which a lubricating layer having furthermore superior adhesion properties with respect to a protective layer can be formed is obtained.

In R¹ and R⁷, when a linking group between a carbon atom to which a terminal polar group is bound and a carbon atom to which a polar group adjacent to the terminal polar group is bound do not contain an oxygen atom (for example, in cases of terminal groups represented by Formulae (4-1) and (4-5) below), in any case where R¹ and R⁷ each have two or three polar groups, the above-described linking group preferably has a linear structure containing 1 to 4 carbon atoms to which no polar group is bound. In a case where the above-described linking group does not contain an oxygen atom and has a linear structure containing one or more carbon atoms to which no polar group is bound, a fluorine-containing ether compound having favorable hydrophobicity is obtained. In addition, if the above-described linking group has a linear structure containing 4 or less carbon atoms, problems are not caused in adhesion properties with respect to the protective layer due to a too hydrophobic linking group. In addition, since the linking group between a carbon atom to which a terminal polar group is bound and a carbon atom to which a polar group adjacent to the terminal polar group is bound does not contain an oxygen atom, intramolecular interaction is small and intramolecular aggregation is less likely to occur. For this reason, a fluorine-containing ether compound with which a lubricating layer having furthermore superior adhesion properties with respect to a protective layer can be formed is obtained. As a result, a lubricating layer containing a fluorine-containing ether compound in which the above-described linking group in R¹ and R⁷ has a linear structure containing the number of carbon atoms within the above-described range can have excellent adhesion properties with respect to a protective layer and can prevent water from intruding due to appropriate hydrophobicity, thereby obtaining a high effect of suppressing corrosion of a magnetic recording medium.

In the fluorine-containing ether compound represented by Formula (1), R¹ and R⁷ may be the same as or different from each other, and are preferably the same as each other. If R¹ and R⁷ are the same as each other, a fluorine-containing ether compound which is likely to wet and spread evenly on the protective layer and from which a lubricating layer having a uniform film thickness is likely to be obtained is obtained. As a result, the lubricating layer containing this fluorine-containing ether compound has a favorable coating rate, which is preferable. In addition, in the case where R¹ and R⁷ are the same as each other, a fluorine-containing ether compound can be produced efficiently through fewer production steps compared to a case where R¹ and R⁷ are different from each other.

In the fluorine-containing ether compound represented by Formula (1), it is preferable that —R¹ and —R⁷ each independently be a terminal group represented by any of Formulae (4-1) to (4-5) below. In a case where —R¹ and —R⁷ are terminal groups represented by any of Formulae (4-1) to (4-5), —R¹ and —R⁷ each contain two or three hydroxyl groups, and a linking group between a carbon atom to which a terminal hydroxyl group is bound and a carbon atom to which a hydroxyl group adjacent to the terminal hydroxyl group is bound has a linear structure with an appropriate number of carbon atoms. Accordingly, in the case where —R¹ and —R⁷ are terminal groups represented by Formulae (4-1) to (4-5), a fluorine-containing ether compound having appropriate hydrophobicity is obtained.

Furthermore, in a case where —R¹ and —R⁷ are terminal groups represented by any of Formulae (4-1), (4-2), (4-4), or (4-5) and each contain three hydroxyl groups, a linking group between a carbon atom to which a hydroxyl group arranged on the perfluoropolyether chain side is bound and a carbon atom to which a hydroxyl group adjacent to that hydroxyl group is bound also has a linear structure with an appropriate number of carbon atoms. Accordingly, a fluorine-containing ether compound is obtained with which a lubricating layer which has superior adhesion properties with respect to a protective layer, has appropriate hydrophobicity, and is highly effective in suppressing corrosion of a magnetic recording medium can be formed.

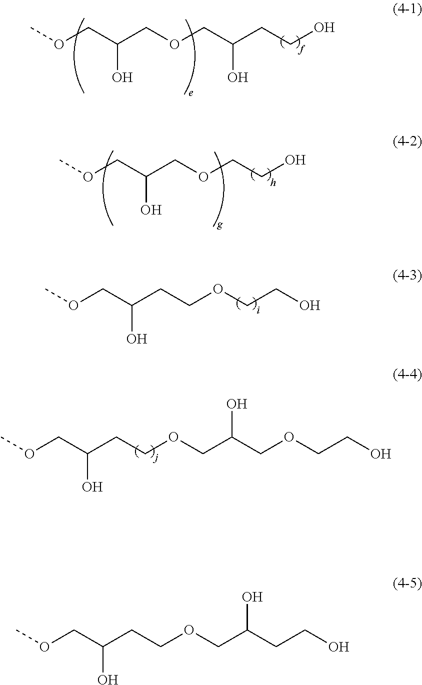

(In Formula (4-1), e is an integer of 0 to 1 and f is an integer of 1 to 4.)

(In Formula (4-2), g is an integer of 1 to 2 and h is an integer of 1 to 3.)

(In Formula (4-3), i is an integer of 1 to 3.)

(In Formula (4-4), j is an integer of 1 to 2.)

In the terminal group represented by Formula (4-1), e is an integer of 0 to 1 and f is an integer of 1 to 4. In the terminal group represented by Formula (4-1), a linking group between a carbon atom to which a terminal hydroxyl group is bound and a carbon atom to which a hydroxyl group adjacent to the terminal hydroxyl group is bound does not contain an oxygen atom. For this reason, appropriate hydrophobicity is exhibited. In addition, since f is 4 or less, problems are not caused in adhesion properties with respect to the protective layer due to a too hydrophobic linking group, thereby exhibiting an excellent effect of suppressing corrosion of a magnetic recording medium.

Furthermore, since the linking group between a carbon atom to which a terminal hydroxyl group is bound and a carbon atom to which a hydroxyl group adjacent to the terminal hydroxyl group is bound does not contain an oxygen atom, intramolecular interaction between the hydroxyl groups contained in the terminal group is small. Accordingly, intramolecular aggregation is less likely to occur, and a lubricating layer having furthermore superior adhesion properties with respect to the protective layer can be formed. In addition, in a case where e is 1, the terminal group represented by Formula (4-1) contains three hydroxyl groups, and therefore superior adhesion properties with respect to the protective layer are exhibited.

In the terminal group represented by Formula (4-2), g is an integer of 1 to 2 and h is an integer of 1 to 3. For this reason, a fluorine-containing ether compound having the terminal group represented by Formula (4-2) has favorable hydrophobicity and exhibits an excellent effect of suppressing corrosion of a magnetic recording medium. In the terminal group represented by Formula (4-2), a linking group between a carbon atom to which a terminal hydroxyl group is bound and a carbon atom to which a hydroxyl group adjacent to the terminal hydroxyl group is bound contains an oxygen atom. Since h is 1 to 3 in the terminal group represented by Formula (4-2), molecular mobility is appropriate. Accordingly, intramolecular aggregation of the hydroxyl groups contained in the terminal group is less likely to occur, and adhesion properties with respect to the protective layer are furthermore superior. In addition, in a case where g is 2, the terminal group represented by Formula (4-2) contains three hydroxyl groups, and therefore superior adhesion properties with respect to the protective layer are furthermore exhibited.

In the terminal group represented by Formula (4-3), i is an integer of 1 to 3. For this reason, a fluorine-containing ether compound containing the terminal group represented by Formula (4-3) has favorable hydrophobicity and exhibits an excellent effect of suppressing corrosion of a magnetic recording medium. In the terminal group represented by Formula (4-3), a linking group between a carbon atom to which a terminal hydroxyl group is bound and a carbon atom to which a hydroxyl group adjacent to the terminal hydroxyl group is bound contains an oxygen atom. Since i is 1 to 3 in the terminal group represented by Formula (4-3), molecular mobility is appropriate. Accordingly, intramolecular aggregation of the hydroxyl groups contained in the terminal group is less likely to occur, and adhesion properties with respect to the protective layer are furthermore superior.

In the terminal group represented by Formula (4-4), j is an integer of 1 to 2. For this reason, a fluorine-containing ether compound containing the terminal group represented by Formula (4-4) has favorable hydrophobicity and exhibits an excellent effect of suppressing corrosion of a magnetic recording medium. The terminal group represented by Formula (4-4) contains three hydroxyl groups, and therefore excellent adhesion properties with respect to the protective layer are exhibited.

In the terminal group represented by Formula (4-5), since a linking group between a carbon atom to which a terminal hydroxyl group is bound and a carbon atom to which a hydroxyl group adjacent to the terminal hydroxyl group is bound does not contain an oxygen atom, favorable hydrophobicity is exhibited. Accordingly, a lubricating layer containing a fluorine-containing ether compound having the terminal group represented by Formula (4-5) exhibits an excellent effect of suppressing corrosion of a magnetic recording medium. In addition, the terminal group represented by Formula (4-5) contains three hydroxyl groups, and therefore excellent adhesion properties with respect to the protective layer are exhibited.

(R³ and R⁵)

In the fluorine-containing ether compound represented by Formula (1), R³ is a linking group represented by Formula (2) below. In the linking group represented by Formula (2), the rightmost oxygen atom in the parentheses of Formula (2) is bound to a methylene group (—CH₂—) adjacent to R⁴. In addition, in the fluorine-containing ether compound represented by Formula (1), R⁵ is a linking group represented by Formula (3) below. In the linking group represented by Formula (3), the leftmost oxygen atom in the parentheses of Formula (3) is bound to a methylene group (—CH₂—) adjacent to R⁴.

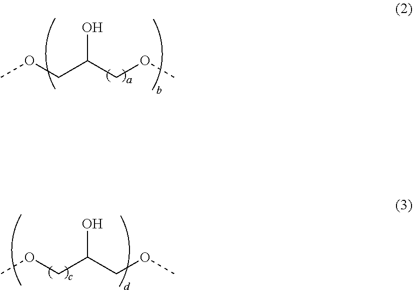

(In Formula (2), a is an integer of 1 to 3 and b is an integer of 1 to 2, and in Formula (3), c is an integer of 1 to 3 and d is an integer of 1 to 2, provided that a in Formula (2) and c in Formula (3) are not 1 at the same time.)

a in Formula (2) is an integer of 1 to 3, and c in Formula (3) is an integer of 1 to 3. a in Formula (2) and c in Formula (3) are not 1 at the same time. That is, at least one of a and c is an integer of 2 to 3. Accordingly, the fluorine-containing ether compound represented by Formula (1) has a larger number of carbon atoms contained in the linking groups represented by R³ and/or R⁵ and more favorable hydrophobicity than a compound in which a in Formula (2) representing R³ is 1 and c in Formula (3) representing R⁵ is 1.

In the fluorine-containing ether compound represented by Formula (1), it is preferable that a in Formula (2) be an integer of 2 to 3 and c in Formula (3) be an integer of 2 to 3. In this case, the fluorine-containing ether compound represented by Formula (1) has better hydrophobicity, and a lubricating layer containing this fluorine-containing ether compound exhibits a superior effect of suppressing corrosion of a magnetic recording medium.

b in Formula (2) is an integer of 1 to 2, and d in Formula (3) is an integer of 1 to 2. The hydroxyl groups in Formulae (2) and (3) improve adhesion properties of the lubricating layer containing the fluorine-containing ether compound with respect to the protective layer. b in Formula (2) and d in Formula (3) are preferably 1 to obtain an easy-to-synthesize fluorine-containing ether compound.

In the fluorine-containing ether compound represented by Formula (1), R³ is the linking group represented by Formula (2) and R⁵ is the linking group represented by Formula (3). Accordingly, oxygen atoms placed at both end portions of each of R³ and R⁵ are respectively bound to methylene groups (—CH₂—) placed on both sides of each of R³ and R⁵ to form ether bonds (—O—). These four ether bonds formed in this manner impart moderate flexibility to the fluorine-containing ether compound represented by Formula (1) and increase the affinity between the protective layer and the hydroxyl groups in R³ and R⁵.

(R², R⁴, and R⁶)

R², R⁴, and R⁶ in the fluorine-containing ether compound represented by Formula (1) are the same or different perfluoropolyether chains (PFPE chains). Due to the PFPE chains represented by R², R⁴, and R⁶, in a case where a lubricant containing the fluorine-containing ether compound of the present embodiment is applied onto a protective layer to form a lubricating layer, the surface of the protective layer is covered and lubricity is imparted to the lubricating layer to reduce frictional force between a magnetic head and the protective layer. In addition, since the PFPE chains have low surface energy, water resistance is imparted to the lubricating layer containing the fluorine-containing ether compound of the present embodiment and the corrosion resistance of the magnetic recording medium on which the lubricating layer is provided is improved.

R², R⁴, and R⁶ are PFPE chains and can be appropriately selected depending on the performance and the like required of a lubricant containing a fluorine-containing ether compound. Examples of PFPE chains include PFPE chains such as a perfluoromethylene oxide polymer, a perfluoroethylene oxide polymer, a perfluoro-n-propylene oxide polymer, a perfluoroisopropylene oxide polymer, and copolymers thereof.

The PFPE chains may have a structure represented by Formula (Rf) below derived from a perfluoroalkylene oxide polymer or copolymer, for example.

—(CF₂)_{w1}O(CF₂O)_{w2}(CF₂CF₂O)_{w3}(CF₂CF₂CF₂O)_{w4}(CF₂CF₂CF₂CF₂O)_{w5}(CF₂)_{w6}— (Rf)

(In Formula (Rf), w2, w3, w4, and w5 indicate the average degree of polymerization and each independently represent 0 to 20, provided that all of w2, w3, w4, and w5 are not 0 at the same time, w1 and w6 are average values indicating the number of —CF₂— and each independently represent 1 to 3, and the arrangement sequence of repeating units in Formula (Rf) is not particularly limited.)

In Formula (Rf), w2, w3, w4, and w5 indicate the average degree of polymerization and each independently represent 0 to 20, preferably 0 to 15, more preferably 0 to 10.

In Formula (Rf), w1 and w6 are average values indicating the number of —CF₂— and each independently represent 1 to 3. w1 and w6 are determined according to the structure or the like of the repeating units arranged at the end portions of the chain structure in the polymer represented by Formula (Rf).

(CF₂O), (CF₂CF₂O), (CF₂CF₂CF₂O), and (CF₂CF₂CF₂CF₂O) in Formula (Rf) are repeating units. The arrangement sequence of the repeating units in Formula (Rf) are not particularly limited. In addition, the number of types of repeating units in Formula (Rf) are not particularly limited.

It is preferable that R², R⁴, and R⁶ in Formula (1) each independently be, for example, a PFPE chain represented by Formula (Rf-1) below.

(CF₂)_{w7}O—(CF₂CF₂O)_{w8}—(CF₂CF₂CF₂O)_{w9}—(CF₂)_{w10}— (Rf-1)

(In Formula (Rf-1), w8 and w9 indicate the average degree of polymerization and each independently represent 0.1 to 20, and w7 and w10 are average values indicating the number of —CF₂— and each independently represent 1 to 2.)

The arrangement sequence of (CF₂CF₂O) and (CF₂CF₂CF₂O) which are repeating units in Formula (Rf-1) is not particularly limited. Formula (Rf-1) may include any of a random copolymer, a block copolymer, and an alternating copolymer composed of the monomer units (CF₂CF₂O) and (CF₂CF₂CF₂O). In Formula (Rf-1), w8 and w9 indicate the average degree of polymerization and each independently represent 0.1 to 20, preferably 0.1 to 15, more preferably 1 to 10. In Formula (Rf-1), w7 and w10 are average values indicating the number of —CF₂— and each independently represent 1 to 2. w7 and w10 are determined according to the structure or the like of the repeating units arranged at the end portions of the chain structure in the polymer represented by Formula (Rf-1).

It is also preferable that R², R⁴, and R⁶ in Formula (1) each independently be any of Formulae (5) to (9) below. The arrangement sequence of (CF₂CF₂O) and (CF₂O) which are repeating units in Formula (5) is not particularly limited. Formula (5) may include any of a random copolymer, a block copolymer, and an alternating copolymer composed of the monomer units (CF₂—CF₂—O) and (CF₂—O).

CF₂O—(CF₂CF₂O)ₖ—(CF₂O)ₗ—CF₂— (5)

(In Formula (5), k and l indicate the average degree of polymerization and each independently represent 0.1 to 20.)

CF₂O—(CF₂CF₂O)ₘ—CF₂— (6)

(In Formula (6), m indicates the average degree of polymerization and represents 0.1 to 20.)

CF₂CF₂O—(CF₂CF₂CF₂O)ₙ—CF₂CF₂— (7)

(In Formula (7), n indicates the average degree of polymerization and represents 0.1 to 20.)

CF₂CF₂CF₂O—(CF₂CF₂CF₂CF₂O)ₒ—CF₂CF₂CF₂— (8)

(In Formula (8), o indicates the average degree of polymerization and represents 0.1 to 10.)

CF(CF₃)O—(CF₂CF(CF₃)O)ₚ—CF(CF₃)— (9)

(In Formula (9), p indicates the average degree of polymerization and represents 0.1 to 20.)

k and l indicating average degrees of polymerization in Formula (5) are each 0.1 to 20, m indicating the average degree of polymerization in Formula (6) is 0.1 to 20, n indicating the average degree of polymerization in Formula (7) is 0.1 to 20, o indicating the average degree of polymerization in Formula (8) is 0.1 to 10, and p indicating the average degree of polymerization in Formula (9) is 0.1 to 20. k, l, m, n, o, and p can be arbitrarily selected within the above-described ranges. If k, l, m, n, o, and p are each 0.1 or more, a fluorine-containing ether compound with which a lubricating layer capable of furthermore suppressing corrosion of a magnetic recording medium can be obtained is obtained. In addition, if k, l, m, n, and p are each 20 or less and o is 10 or less, the viscosity of a fluorine-containing ether compound does not become too high, and a lubricant containing this fluorine-containing ether compound becomes easy to apply, which is preferable. All of k, l, m, n, o, and p indicating the average degree of polymerization are preferably 1 to 10 and more preferably 2 to 8 to obtain a fluorine-containing ether compound which is likely to wet and spread on a protective layer and from which a lubricating layer having a uniform film thickness is likely to be obtained. k, l, m, n, o, and p may be, for example, 0.5 to 9, 1 to 8, 2 to 7, 3 to 6, or 4 to 5.

In the case where R², R⁴, and R⁶ in Formula (1) is any of Formulae (5) to (9), a fluorine-containing ether compound is easily synthesized, which is preferable. In a case where R², R⁴, and R⁶ are any of Formulae (5) to (7), a raw material is readily available, which is preferable.

In addition, in the case where R², R⁴, and R⁶ are any of Formulae (5) to (9), the ratio of the number of oxygen atoms (the number of ether bonds (—O—)) to the number of carbon atoms in the perfluoropolyether chains is appropriate. For this reason, a fluorine-containing ether compound with moderate hardness is obtained. Accordingly, a fluorine-containing ether compound applied onto a protective layer is less likely to be aggregated on the protective layer, and a lubricating layer having an even thinner thickness at a sufficient coating rate can be formed.

In the fluorine-containing ether compound represented by Formula (1), the PFPE chains represented by R², R⁴, and R⁶ may all be the same as or different from each other, or may be the same only in part. In the present embodiment, the same PFPE chains include a case where PFPE chains have the same structure (repeating unit) but have different average degrees of polymerization.

In the fluorine-containing ether compound represented by Formula (1), R² and R⁶ are preferably the same as each other to facilitate synthesis. In the case where R² is the same as R⁶, it is more preferable that R³ be the same as R⁵ and R¹ be the same as R⁷. Such a fluorine-containing ether compound can be easily and efficiently produced with a small number of production steps. In the present specification, R³ and R⁵ being the same as each other mean that atoms contained in R³ and atoms contained in R⁵ are symmetrically arranged with respect to the perfluoropolyether chain (R⁴) placed at the center of the fluorine-containing ether compound represented by Formula (1). That is, this means that a in Formula (2) is the same as c in Formula (3) and b in Formula (2) is the same as d in Formula (3).

It is preferable that the fluorine-containing ether compound represented by Formula (1) be specifically any compound represented by Formulae (A) to (T) below.

Since ra, qb, sc, tc, rd, qe, qf, sg, tg, rh, qi, sj, tj, qk, rk, sl, tl, rl, qm, rm, qn, rn, qo, ro, qp, sp, tp, qq, sq, tq, sr, tr, rr, qs, ss, ts, st, tt, and rt in Formulae (A) to (T) are values indicating the average degree of polymerization, these are not necessarily integers.

In all the compounds represented by Formulae (A) to (T) below, R¹ and R⁷ are the same as each other.

In all the compounds represented by Formulae (A) to (J) below, R², R⁴, and R⁶ are the same as each other. In all the compounds represented by Formulae (K) to (T) below, R² and R⁶ are the same as each other.

In all the compounds represented by Formulae (A) to (G), (K), (L), (N) to (P), (R), and (S), R³ and R⁵ are the same as each other, a and b in Formula (2) are respectively 2 and 1, and c and d in Formula (3) are respectively 2 and 1.

In all the compounds represented by Formulae (H) to (J), (M), (Q), and (T), R³ and R⁵ are the same as each other, a and b in Formula (2) are respectively 3 and 1, and c and d in Formula (3) are respectively 3 and 1.

In the compound represented by Formula (A) below, R¹ and R⁷ are terminal groups represented by Formula (4-2), g and h in Formula (4-2) are respectively 1 and 2, and R², R⁴, and R⁶ are PFPE chains represented by Formula (6).

In the compound represented by Formula (B) below, R¹ and R⁷ are terminal groups represented by Formula (4-2), g and h in Formula (4-2) are 2, and R², R⁴, and R⁶ are PFPE chains represented by Formula (7).

In the compound represented by Formula (C) below, R¹ and R⁷ are terminal groups represented by Formula (4-1), e and f in Formula (4-1) are respectively 0 and 1, and R², R⁴, and R⁶ are PFPE chains represented by Formula (5).

In the compound represented by Formula (D) below, R¹ and R⁷ are terminal groups represented by Formula (4-5), and R², R⁴, and R⁶ are PFPE chains represented by Formula (6).

In the compound represented by Formula (E) below, R¹ and R⁷ are terminal groups represented by Formula (4-2), g and h in Formula (4-2) are 1, and R², R⁴, and R⁶ are PFPE chains represented by Formula (7).

In the compound represented by Formula (F) below, R¹ and R⁷ are terminal groups represented by Formula (4-3), i in Formula (4-3) is 1, and R², R⁴, and R⁶ are PFPE chains represented by Formula (7).

In the compound represented by Formula (G) below, R¹ and R⁷ are terminal groups represented by Formula (4-1), e and f in Formula (4-1) are 1, and R², R⁴, and R⁶ are PFPE chains represented by Formula (5).

In the compound represented by Formula (H) below, R¹ and R⁷ are terminal groups represented by Formula (4-1), e and f in Formula (4-1) are respectively 0 and 2, and R², R⁴, and R⁶ are PFPE chains represented by Formula (6).

In the compound represented by Formula (I) below, R¹ and R⁷ are terminal groups represented by Formula (4-4), j in Formula (4-4) is 2, and R², R⁴, and R⁶ are PFPE chains represented by Formula (7).

In the compound represented by Formula (J) below, R¹ and R⁷ are terminal groups represented by Formula (4-2), g and h in Formula (4-2) are respectively 2 and 1, and R², R⁴, and R⁶ are PFPE chains represented by Formula (5).

In the compound represented by Formula (K) below, R¹ and R⁷ are terminal groups represented by Formula (4-1), e and f in Formula (4-1) are 1, R² and R⁶ are PFPE chains represented by Formula (7), and R⁴ is a PFPE chain represented by Formula (6).

In the compound represented by Formula (L) below, R¹ and R⁷ are terminal groups represented by Formula (4-4), j in Formula (4-4) is 1, R² and R⁶ are PFPE chains represented by Formula (5), and R⁴ is a PFPE chain represented by Formula (6).

In the compound represented by Formula (M) below, R¹ and R⁷ are terminal groups represented by Formula (4-2), g and h in Formula (4-2) are respectively 1 and 2, R² and R⁶ are PFPE chains represented by Formula (7), and R⁴ is a PFPE chain represented by Formula (6).

In the compound represented by Formula (N) below, R¹ and R⁷ are terminal groups represented by Formula (4-2), g and h in Formula (4-2) are 2, R² and R⁶ are PFPE chains represented by Formula (6), and R⁴ is a PFPE chain represented by Formula (7).

In the compound represented by Formula (O) below, R¹ and R⁷ are terminal groups represented by Formula (4-5), R² and R⁶ are PFPE chains represented by Formula (6), and R⁴ is a PFPE chain represented by Formula (7).

In the compound represented by Formula (P) below, R¹ and R⁷ are terminal groups represented by Formula (4-2), g and h in Formula (4-2) are respectively 1 and 3, R² and R⁶ are PFPE chains represented by Formula (5), and R⁴ is a PFPE chain represented by Formula (7).

In the compound represented by Formula (Q) below, R¹ and R⁷ are terminal groups represented by Formula (4-2), g and h in Formula (4-2) are 1, R² and R⁶ are PFPE chains represented by Formula (5), and R⁴ is a PFPE chain represented by Formula (7).

In the compound represented by Formula (R) below, R¹ and R⁷ are terminal groups represented by Formula (4-2), g and h in Formula (4-2) are respectively 2 and 1, R² and R⁶ are PFPE chains represented by Formula (6), and R⁴ is a PFPE chain represented by Formula (5).

In the compound represented by Formula (S) below, R¹ and R⁷ are terminal groups represented by Formula (4-1), e and f in Formula (4-1) are respectively 0 and 3, R² and R⁶ are PFPE chains represented by Formula (7), and R⁴ is a PFPE chain represented by Formula (5).

In the compound represented by Formula (T) below, R¹ and R⁷ are terminal groups represented by Formula (4-1), e and f in Formula (4-1) are respectively 1 and 2, R² and R⁶ are PFPE chains represented by Formula (6), and R⁴ is a PFPE chain represented by Formula (5).

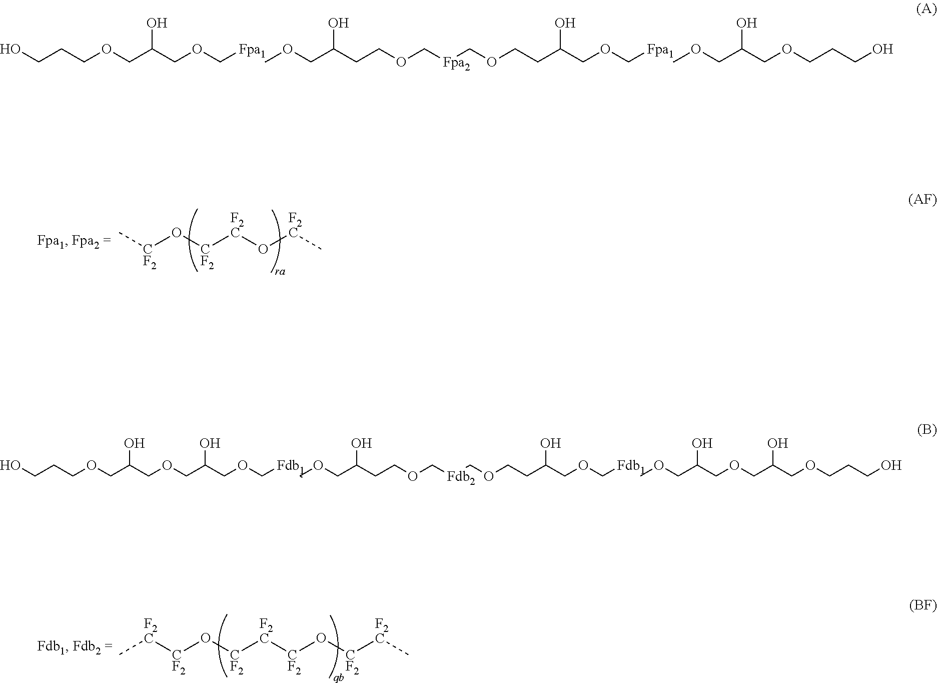

(Fpa₁ and Fpa₂ in Formula (A) are represented by Formula (AF), ra in Fpa₁ and Fpa₂ indicates the average degree of polymerization and represents 0.1 to 20, and ra in Ppa₁ and ra in Ppa₂ may be the same as or different from each other.)

(Fdb₁ and Fdb₂ in Formula (B) are represented by Formula (BF), qb in Fdb₁ and Fdb₂ indicates the average degree of polymerization and represents 0.1 to 20, and qb in Fdb₁ and qb in Fdb₂ may be the same as or different from each other.)

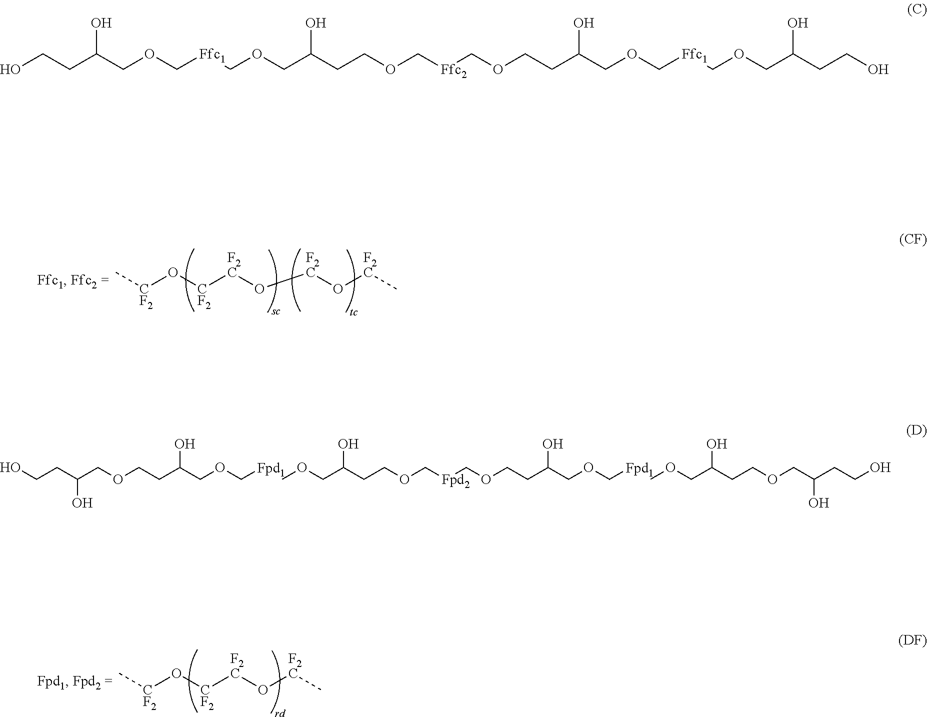

(Ffc₁ and Ffc₂ in Formula (C) are represented by Formula (CF), sc and tc in Ffc₁ and Ffc₂ indicate the average degree of polymerization and represent 0.1 to 20, and sc and tc in Ffc₁ and sc and tc in Ffc₂ may be the same as or different from each other.)

(Fpd₁ and Fpd₂ in Formula (D) are represented by Formula (DF), rd in Fpd₁ and Fpd₂ indicates the average degree of polymerization and represents 0.1 to 20, and rd in Fpd₁ and rd in Fpd₂ may be the same as or different from each other.)

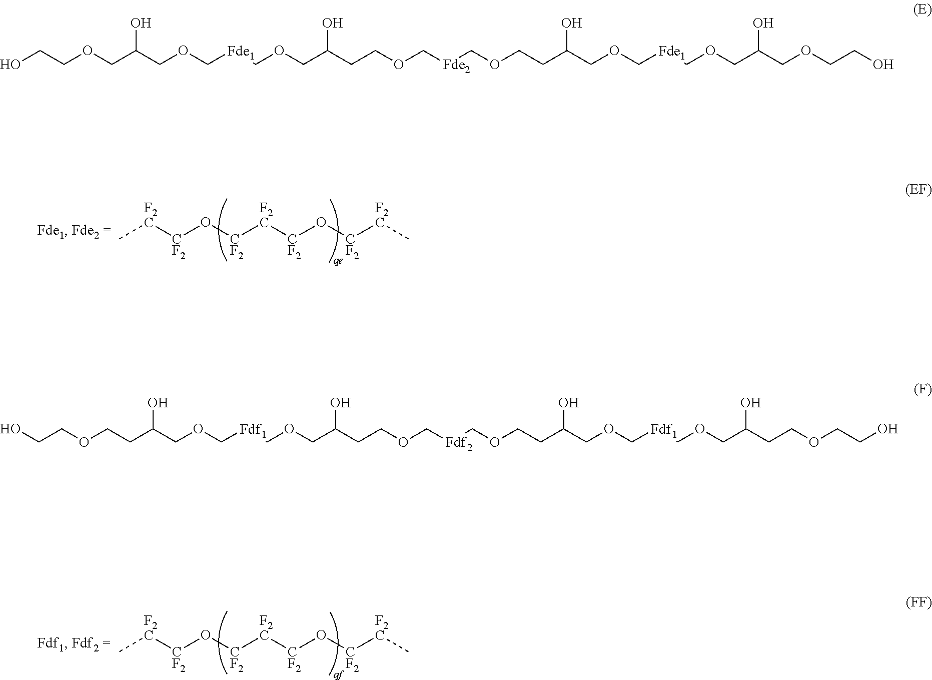

(Fde₁ and Fde₂ in Formula (E) are represented by Formula (EF), qe in Fde₁ and Fde₂ indicates the average degree of polymerization and represents 0.1 to 20, and qe in Fde₁ and qe in Fde₂ may be the same as or different from each other.)

(Fdf₁ and Fdf₂ in Formula (F) are represented by Formula (FF), qf in Fdf₁ and Fdf₂ indicates the average degree of polymerization and represents 0.1 to 20, and qf in Fdf₁ and qf in Fdf₂ may be the same as or different from each other.)

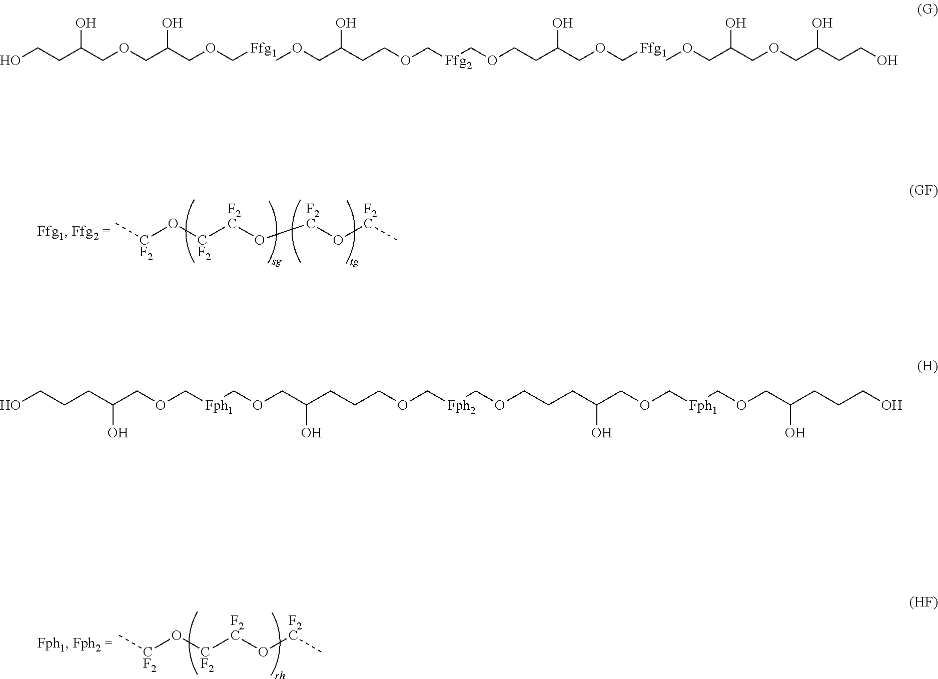

(Ffg₁ and Ffg₂ in Formula (G) are represented by Formula (GF), sg and tg in Ffg₁ and Ffg₂ indicate the average degree of polymerization and represent 0.1 to 20, and sg and tg in Ffg₁ and sg and tg in Ffg₂ may be the same as or different from each other.)

(Fph₁ and Fph₂ in Formula (H) are represented by Formula (HP), rh in Fph₁ and Fph₂ indicates the average degree of polymerization and represents 0.1 to 20, and rh in Fph₁ and rh in Fph₂ may be the same as or different from each other.)

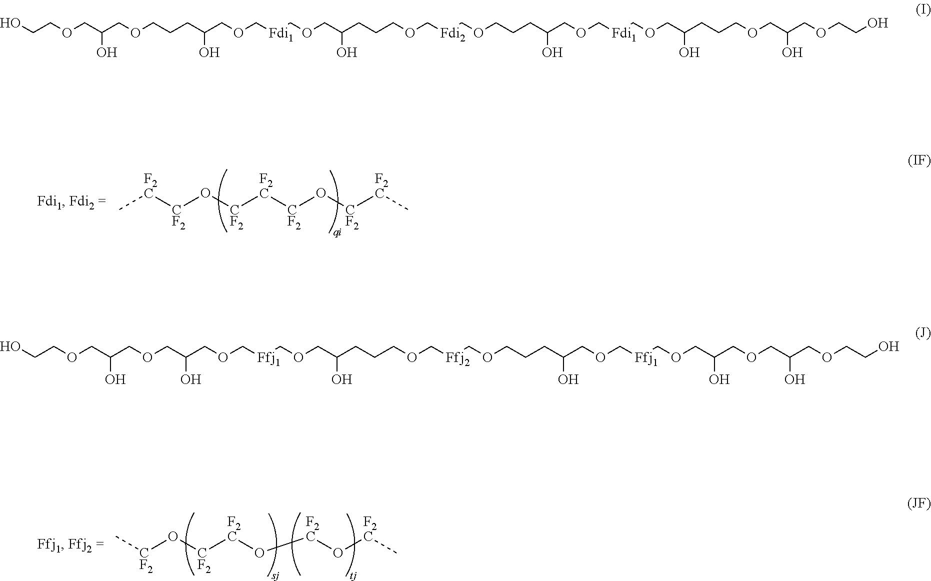

(Fdi₁ and Fdi₂ in Formula (I) are represented by Formula (IF), qi in Fdi₁ and Fdi₂ indicates the average degree of polymerization and represents 0.1 to 20, and qi in Fdi₁ and qi in Fdi₂ may be the same as or different from each other.)

(Ffj₁ and Ffj₂ in Formula (J) are represented by Formula (JF), sj and tj in Ffj₁ and Ffj₂ indicate the average degree of polymerization and represent 0.1 to 20, and sj and tj in Ffj₁ and sj and tj in Ffj₂ may be the same as or different from each other.)

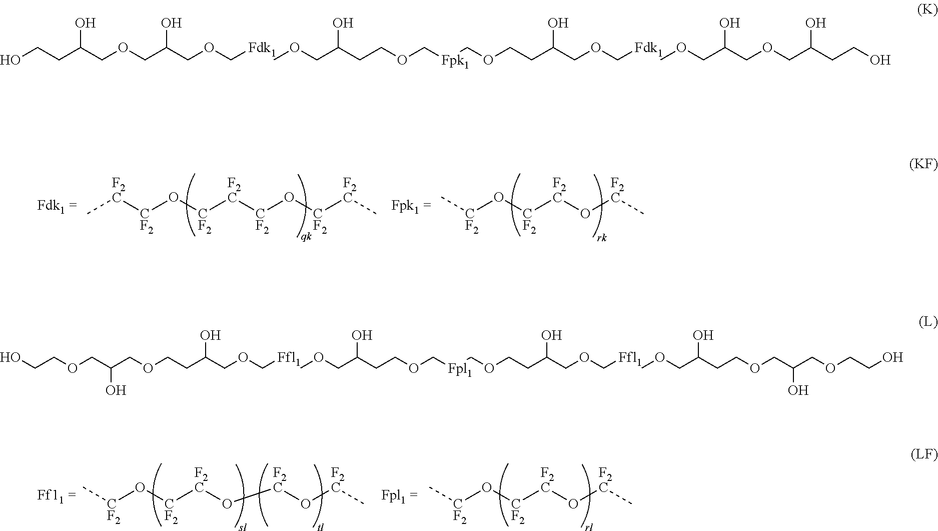

(Fdk₁ and Fpk₁ in Formula (K) are represented by Formula (KF), qk in Fdk₁ indicates the average degree of polymerization and represents 0.1 to 20, and rk in Fpk₁ indicates the average degree of polymerization and represents 0.1 to 20.)

(Ffl₁ and Fpl₁ in Formula (L) are represented by Formula (LF), si and tl in Ffl₁ indicate the average degree of polymerization and represent 0.1 to 20, and rl in Fpl₁ indicates the average degree of polymerization and represents 0.1 to 20.)

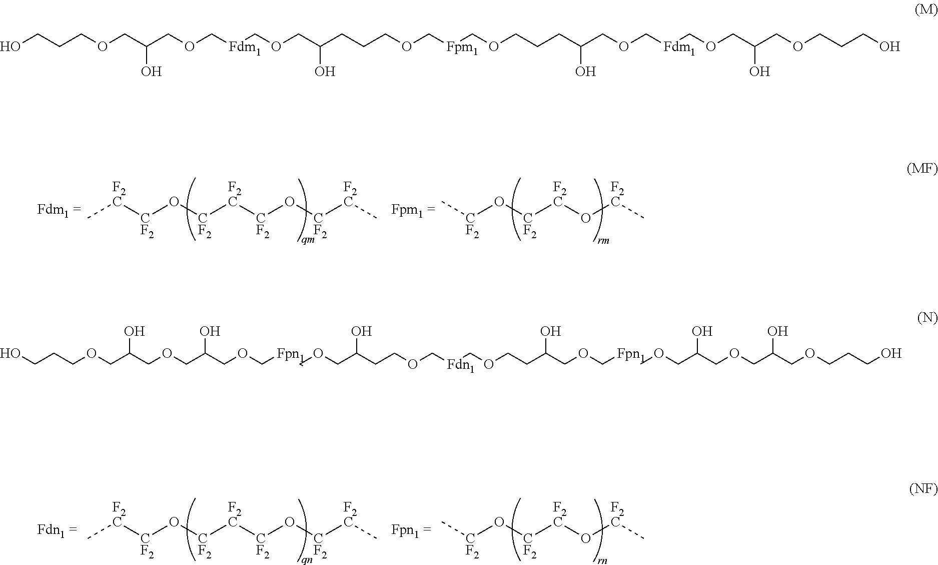

(Fdm₁ and Fpm₁ in Formula (M) are represented by Formula (MF), qm in Fdm₁ indicates the average degree of polymerization and represents 0.1 to 20, and rm in Fpm₁ indicates the average degree of polymerization and represents 0.1 to 20.)

(Fdn₁ and Fpn₁ in Formula (N) are represented by Formula (NF), qn in Fdn₁ indicates the average degree of polymerization and represents 0.1 to 20, and m in Fpn₁ indicates the average degree of polymerization and represents 0.1 to 20.)

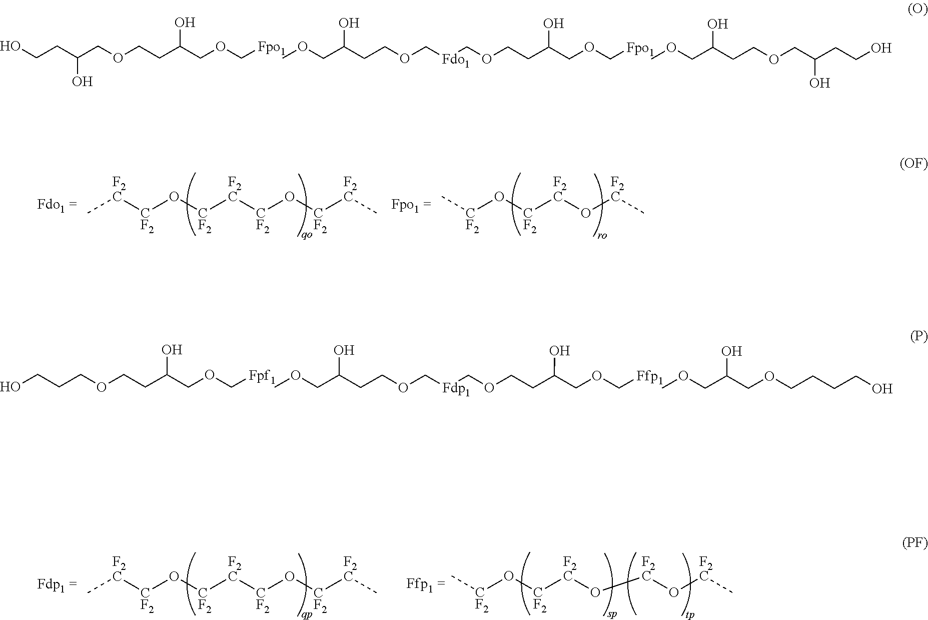

(Fdo₁ and Fpo₁ in Formula (O) are represented by Formula (OF), qo in Fdo₁ indicates the average degree of polymerization and represents 0.1 to 20, and ro in Fpo₁ indicates the average degree of polymerization and represents 0.1 to 20.)

(Fdp₁ and Ffp₁ in Formula (P) are represented by Formula (PF), qp in Fdp₁ indicates the average degree of polymerization and represents 0.1 to 20, and sp and tp in Ffp₁ indicate the average degree of polymerization and represent 0.1 to 20.)

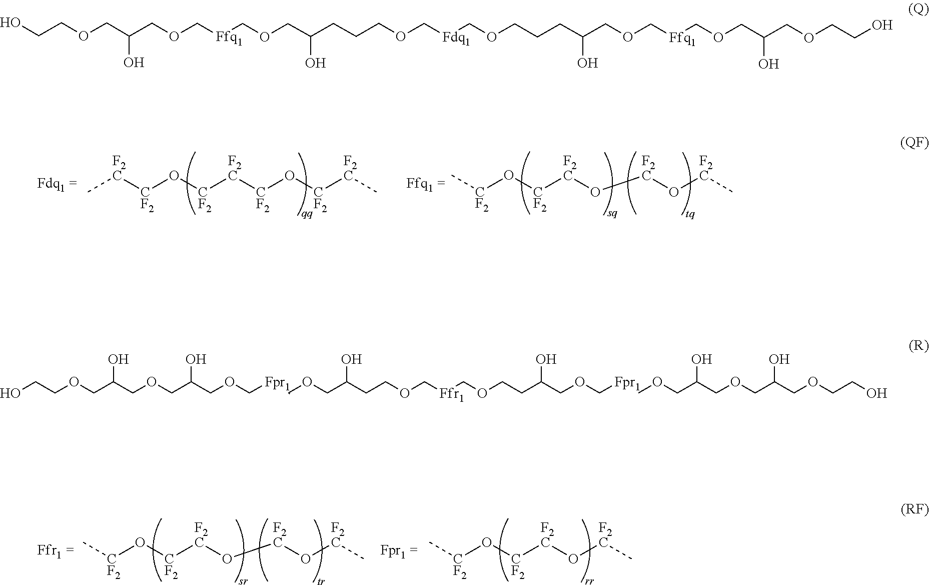

(Fdq₁ and Ffq₁ in Formula (Q) are represented by Formula (QF), qq in Fdq₁ indicates the average degree of polymerization and represents 0.1 to 20, and sq and tq in Ffq₁ indicate the average degree of polymerization and represent 0.1 to 20.)

(Ffr₁ and Fpr₁ in Formula (R) are represented by Formula (RF), ar and tr in Ffr₁ indicate the average degree of polymerization and represent 0.1 to 20, and rr in Fpr₁ indicates the average degree of polymerization and represents 0.1 to 20.)

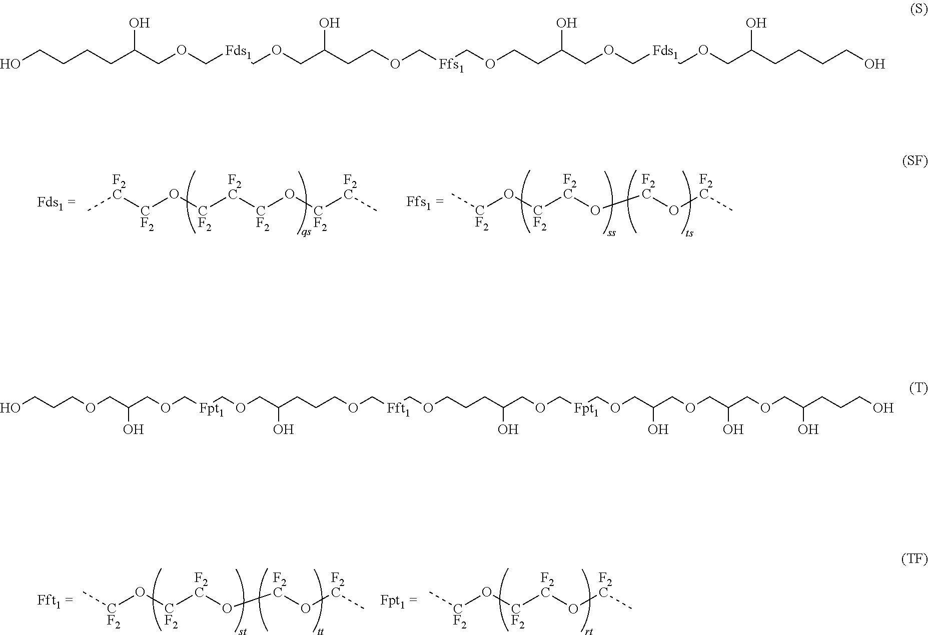

(Fds₁ and Ffs₁ in Formula (S) are represented by Formula (SF), qs in Fds₁ indicates the average degree of polymerization and represents 0.1 to 20, and ss and ts in Ffs₁ indicate the average degree of polymerization and represent 0.1 to 20.)

(Fft₁ and Fpt₁ in Formula (T) are represented by Formula (TF), st and tt in Fft₁ indicate the average degree of polymerization and represent 0.1 to 20, and rt in Fpt₁ indicates the average degree of polymerization and represents 0.1 to 20.)

When the compound represented by Formula (1) is any of compounds represented by Formulae (A) to (T), it is preferable because the procurement of raw materials is easy and a lubricating layer capable of suppressing corrosion of a magnetic recording medium can be formed even if the lubricating layer has a thin thickness.

When the compound represented by Formula (1) is any of the compounds represented by Formulae (B), (D), and (F) to (T), it is particularly preferable because it is possible to form a lubricating layer highly effective in suppressing corrosion of a magnetic recording medium.

In the fluorine-containing ether compound of the present embodiment, the number average molecular weight (Mn) of the compound is preferably within a range of 500 to 10,000, more preferably within a range of 700 to 7,000, and particularly preferably within a range of 1,000 to 5,000. If the number average molecular weight thereof is 500 or more, a lubricant containing the fluorine-containing ether compound of the present embodiment hardly evaporates, whereby the lubricant can be prevented from evaporating and transferring to a magnetic head. In addition, if the number average molecular weight is 10,000 or less, the fluorine-containing ether compound has an appropriate viscosity, and a thin lubricating layer can be easily formed by applying a lubricant containing this fluorine-containing ether compound. If the number average molecular weight is 5,000 or less, in a case where the fluorine-containing ether compound is applied to a lubricant, the viscosity of the lubricant becomes appropriate for handling, which is more preferable.

The number average molecular weight (Mn) of the fluorine-containing ether compound is a value measured by ¹H-NMR and ¹⁹F-NMR with AVANCE III400 manufactured by Bruker BioSpin Group. In the nuclear magnetic resonance (NMR) measurement, a sample is diluted with a single or mixed solvent of hexafluorobenzene, acetone-d, tetrahydrofuran-d, and the like and used in the measurement. As the reference of the ¹⁹F-NMR chemical shift, the peak of hexafluorobenzene was set to −164.7 ppm. As the reference of the ¹H-NMR chemical shift, the peak of acetone was set to 2.2 ppm.

### “Production Method”

A method for producing the fluorine-containing ether compound of the present embodiment is not particularly limited, and the fluorine-containing ether compound can be produced using a well-known conventional production method. The fluorine-containing ether compound of the present embodiment can be produced using, for example, a production method shown below.

In the present embodiment, a case of producing a compound having a symmetrical structure around R⁴ will be described as an example of the fluorine-containing ether compound represented by Formula (1). Specifically, a case of producing a compound in which three PFPE chains represented by R², R⁴, and R⁶ in Formula (1) have the same structure, R¹ is the same as R⁷, and R³ is the same as R⁵ will be described as an example.

First, a fluorine compound is prepared in which hydroxymethyl groups (—CH₂OH) are placed at both terminals of a perfluoropolyether chain corresponding to R⁴ in Formula (1). Next, the hydroxyl groups of the hydroxymethyl groups placed at both terminals of the fluorine compound are reacted with a halogen compound having an epoxy group corresponding to R³ (═R⁵) (first reaction). As a result, an intermediate compound 1 having epoxy groups corresponding to R³ (═R⁵) at both terminals of the perfluoropolyether chain corresponding to R⁴ is obtained.

When producing the fluorine-containing ether compound of the present embodiment, examples of halogen compounds having an epoxy group used in the first reaction include epichlorohydrin, epibromohydrin, 2-(2-bromoethyl)oxirane, 2-(2-chloroethyl)oxirane, 2-(3-bromopropyl)oxirane, and 2-(3-chloropropyl)oxirane. In a case where a in Formula (2) representing R³ in Formula (1) and/or c in Formula (3) representing R⁵ are an integer of 2 to 3, as halogen compounds having an epoxy group, 2-(2-bromoethyl)oxirane, 2-(2-chloroethyl)oxirane, 2-(3-bromopropyl)oxirane, and 2-(3-chloropropyl)oxirane can be used, for example.

Subsequently, a fluorine compound is prepared in which hydroxymethyl groups (—CH₂OH) are placed at both terminals of a perfluoropolyether chain corresponding to R² (═R⁴═R⁶) in Formula (1). Then, a hydroxyl group of the hydroxymethyl group placed at one terminal of the fluorine compound is reacted with an epoxy compound having a group corresponding to R¹ (═R⁷) in Formula (1) (second reaction). As a result, an intermediate compound 2 having the group corresponding to R¹ (═R⁷) at one terminal of the perfluoropolyether chain corresponding to R² (═R⁶) is obtained.

The epoxy compound having the group corresponding to R¹ (═R⁷) may be reacted with the above-described fluorine compound after protecting the hydroxyl group with a suitable protecting group.

Epoxy compounds which are used in the second reaction when producing the fluorine-containing ether compound of the present embodiment can be synthesized, for example, by reacting alcohols having a structure corresponding to R¹ (or R⁷) of the fluorine-containing ether compound to be produced with a compound having any epoxy group selected from epichlorohydrin, epibromohydrin, 2-bromoethyloxirane, and allyl glycidyl ether. Such epoxy compounds may be synthesized through a method of oxidizing an unsaturated bond, or commercially available products may be purchased and used.

Thereafter, the hydroxyl group of the hydroxymethyl group placed at one terminal of the intermediate compound 2 is reacted with the epoxy groups placed at both terminals of the intermediate compound 1 (third reaction).

By performing the above-described steps, a compound in which three PFPE chains represented by R², R⁴, and R⁶ in Formula (1) have the same structure, R¹ is the same as R⁷, and R³ is the same as R⁵ can be produced. Here, the order of the first and second reactions may be reversed.

In addition, in a case where, for example, a compound in which R², R⁴, and R⁶ are the same as each other, R³ is the same as R⁵, and R¹ is different from R⁷ is produced as the fluorine-containing ether compound represented by Formula (1), the production method shown below can be used.

That is, in the above-described second reaction, an intermediate compound 2a having a group corresponding to R¹ and an intermediate compound 2b having a group corresponding to R⁷ are synthesized. Thereafter, in the above-described third reaction, the compound can be produced through a method in which the intermediate compound 2a and the intermediate compound 2b are sequentially reacted with the epoxy group corresponding to R³ (═R⁵) placed at each terminal of the intermediate compound 1.

In addition, in a case where, for example, a compound in which R², R⁴, and R⁶ are the same as each other, R¹ is the same as R⁷, and R³ is different from R⁵ is produced as the fluorine-containing ether compound represented by Formula (1), the production method shown below can be used.

That is, the halogen compound having the epoxy group corresponding to R³ and the halogen compound having the epoxy group corresponding to R⁵ are sequentially reacted with the hydroxyl group of the hydroxymethyl group placed at each terminal of the fluorine compound in the above-described first reaction. As a result, an intermediate compound 1a having the epoxy group corresponding to R³ at one end of the perfluoropolyether chain corresponding to R⁴ and the epoxy group corresponding to R⁵ at the other end is obtained. Thereafter, in the third reaction described above, the compound can be produced through a method in which the intermediate compound 1a is used instead of the intermediate compound 1.

In addition, in a case of producing, for example, a compound, in which R¹ is the same as R⁷, R³ is the same as R⁵, and two PFPE chains represented by R² and R⁶ are different from a PFPE chain represented by R⁴, as the fluorine-containing ether compound represented by Formula (1), the compound can be produced through the production method shown below.

That is, the compound can be produced through the same method as described above except that fluorine compounds having different PFPE chains are used in the first reaction and the second reaction described above.

In addition, in a case of producing, for example, a compound, in which R¹ is the same as or different from R⁷, R³ is the same as R⁵, and PFPE chains represented by R², R⁴, and R⁶ are different from each other, as the fluorine-containing ether compound represented by Formula (1), the compound can be produced through the production method shown below.

That is, in the above-described second reaction, two types of fluorine compounds having a different type of PFPE chain as that of the fluorine compound used in the first reaction are used as fluorine compounds. Thus, in the second reaction, an intermediate compound 2c having a group corresponding to R¹ at one terminal of the perfluoropolyether chain corresponding to R² and an intermediate compound 2d having a group corresponding to R⁷ at one terminal of the perfluoropolyether chain corresponding to R⁶ are synthesized. Thereafter, in the above-described third reaction, the compound can be produced through a method in which the intermediate compound 2c and the intermediate compound 2d are sequentially reacted with the epoxy group corresponding to R³ (═R⁵) placed at each terminal of the intermediate compound 1.

Here, the function of a lubricating layer formed on a protective layer using a lubricant containing the fluorine-containing ether compound of the present embodiment will be described.

Examples of the cause of corrosion of a magnetic recording medium include ionic contamination substances present on the surface of the magnetic recording medium. Most of the ionic contamination substances adhere to the magnetic recording medium from outside during the production process of the magnetic recording medium. The ionic contamination substances may also be generated when environmental substances that have intruded into a hard disk drive (magnetic recording/reproducing device) adhere to the magnetic recording medium. Specifically, for example, water containing environmental substances such as ions may adhere to the surface of the magnetic recording medium when the magnetic recording medium and/or hard disk drive are held under high-temperature and high-humidity conditions. When water containing environmental substances such as ions passes through the lubricating layer formed on the surface of the magnetic recording medium, it condenses minute ionic components present under the lubricating layer to generate ionic contamination substances.

Since the fluorine-containing ether compound of the present embodiment is a compound represented by Formula (1), a lubricating layer containing this fluorine-containing ether compound has a high corrosion suppressing effect that prevents contamination substances from intruding into the magnetic recording medium. This effect is obtained by the synergistic effects that the lubricating layer containing the fluorine-containing ether compound of the present embodiment has functions of <1> to <5> described below, and therefore it has excellent adhesion properties with respect to a protective layer, has appropriate hydrophobicity, and is likely to be formed on the protective layer in a uniform coating state.

<1> The above-described lubricating layer adheres closely to a protective layer due to a suitable interaction generated between the protective layer and the lubricating layer due to one or more hydroxyl groups (—OH) in each of R³ and R⁵ in the compound represented by Formula (1) and two or three polar groups each contained in R¹ and R⁷. For this reason, the above-described lubricating layer can prevent contamination substances from intruding into the magnetic recording medium and suppress corrosion of the magnetic recording medium.

<2> In the fluorine-containing ether compound represented by Formula (1), three perfluoropolyether chains (R², R⁴, and R⁶) are each positioned between R¹ and R³, between R³ and R⁵, and between R⁵ and R⁷.

For this reason, the distance between a polar group in R¹ and a hydroxyl group in R³, the distance between hydroxyl groups in R³ and R⁵, the distance between a hydroxyl group in R⁵ and a polar group in R⁷ are all appropriate. Accordingly, both the hydroxyl groups in R³ and R⁵ and the polar groups in R¹ and R⁷ are less likely to be inhibited form binding with active points on a protective layer due to the adjacent polar groups. Accordingly, both the hydroxyl groups in R³ and R⁵ and the polar groups in R¹ and R⁷ are likely to participate in binding with the active points on the protective layer. In other words, all the polar groups in the above-described fluorine-containing ether compound are less likely to be polar groups that do not participate in binding with the active points on the protective layer. As a result, a lubricating layer containing the above-described fluorine-containing ether compound has a reduced number of polar groups that do not participate in binding with the active points on the protective layer and has good adhesion properties with respect to the protective layer.

In addition, since the distance between the hydroxyl group in R³ and the hydroxyl group in R⁵, the distance between the polar group in R¹ and the hydroxyl group in R³, and the distance between the hydroxyl group in R⁵ and the polar group in R⁷ are all appropriate, intramolecular interaction between the polar groups in R³, R⁵, R¹, and R⁷ is small and aggregation is less likely to occur. Accordingly, the fluorine-containing ether compound represented by Formula (1) is likely to wet and spread on the protective layer, and a lubricating layer having a uniform coating state and a high coating rate, and favorable adhesion properties, can be formed on the protective layer.

Furthermore, both end portions of each perfluoropolyether chain (R², R⁴, and R⁶) adhere closely to the protective layer due to the polar groups in any one of R³, R⁵, R¹, and R⁷. For this reason, the fluorine-containing ether compound applied onto the protective layer is less likely to be bulky. Accordingly, the fluorine-containing ether compound represented by Formula (1) is likely to wet and spread on the protective layer, and a lubricating layer having a uniform coating state and a high coating rate, and favorable adhesion properties, can be formed on the protective layer.

<3> The fluorine-containing ether compound represented by Formula (1) has three perfluoropolyether chains (R², R⁴, and R⁶). The perfluoropolyether chains contained in a lubricating layer cover the surface of the protective layer and impart water resistance to the lubricating layer due to their low surface energy.

The fluorine-containing ether compound represented by Formula (1) contained in the lubricating layer undergoes heat-induced molecular motion when held under high-temperature and high-humidity conditions. Water containing environmental substances such as ions is thought to enter through gaps between molecules in molecular motion in the lubricating layer.

Since the lubricating layer containing the fluorine-containing ether compound represented by Formula (1) has appropriate water resistance and hydrophobicity, it prevents water from intruding into the magnetic recording medium through the gaps between the molecules in molecular motion in the lubricating layer and improves corrosion resistance of the magnetic recording medium.

<4> In the fluorine-containing ether compound represented by Formula (1), R³ is a linking group represented by Formula (2), R⁵ is a linking group represented by Formula (3), and a in Formula (2) and c in Formula (3) are not 1 at the same time. That is, the fluorine-containing ether compound represented by Formula (1) has a methylene chain with 2 or 3 carbon atoms between R⁴ and a carbon atom to which hydroxyl groups in Formula (2) and/or Formula (3) are bound. Accordingly, the fluorine-containing ether compound represented by Formula (1) has a larger number of carbon atoms contained in the linking groups represented by R³ and/or R⁵ and more favorable hydrophobicity than a compound in which a in Formula (2) is 1 and c in Formula (3) is 1. As a result, a lubricating layer containing the fluorine-containing ether compound represented by Formula (1) can prevent water from intruding into the magnetic recording medium because water hardly passes therethrough.

<5> In the fluorine-containing ether compound represented by Formula (1), individual polar groups in R¹ and R⁷ are bound to different carbon atoms and the carbon atoms to which the polar groups are bound are bound to each other via a linking group containing a carbon atom to which the polar group is not bound. For this reason, due to the hydrophobicity of the carbon atoms to which the polar groups contained in R¹ and R⁷ are bound and the carbon atoms contained in linking groups in R¹ and R⁷, a lubricating layer containing the fluorine-containing ether compound represented by Formula (1) has appropriate hydrophobicity. As a result, the lubricating layer containing the fluorine-containing ether compound represented by Formula (1) can prevent water from intruding into the magnetic recording medium because water hardly passes therethrough, and can suppress corrosion of the magnetic recording medium.

### [Lubricant for Magnetic Recording Medium]

A lubricant for a magnetic recording medium of the present embodiment contains the fluorine-containing ether compound represented by Formula (1).

The lubricant of the present embodiment can be used after being mixed as necessary with a well-known material that is used as a material for lubricants within the scope not impairing the characteristics due to the incorporation of the fluorine-containing ether compound represented by Formula (1).

Specific examples of well-known materials include FOMBLIN (registered trademark) ZDIAC, FOMBLIN ZDEAL, and FOMBLIN AM-2001 (all manufactured by Solvay Solexis), and Moresco A20H (manufactured by Moresco Corporation). The number average molecular weight of the well-known material that is used by being mixed with the lubricant of the present embodiment is preferably 1,000 to 10,000.

In a case where the lubricant of the present embodiment contains a material other than the fluorine-containing ether compound represented by Formula (1), the content of the fluorine-containing ether compound represented by Formula (1) in the lubricant of the present embodiment is preferably 50 mass % or more and more preferably 70 mass % or more. The content of the fluorine-containing ether compound represented by Formula (1) may be 80 mass % or more or 90 mass % or more.

Since the lubricant of the present embodiment contains the fluorine-containing ether compound represented by Formula (1), a lubricating layer highly effective in suppressing corrosion of a magnetic recording medium can be formed. The lubricating layer consisting of the lubricant of the present embodiment is highly effective in suppressing corrosion of a magnetic recording medium, and therefore can be made thin.

### [Magnetic Recording Medium]

A magnetic recording medium of the present embodiment includes at least a magnetic layer, a protective layer, and a lubricating layer sequentially provided on a substrate.

In the magnetic recording medium of the present embodiment, one or two or more underlayers can be provided as necessary between the substrate and the magnetic layer. In addition, it is also possible to provide an adhesive layer and/or a soft magnetic layer between the underlayer and the substrate.

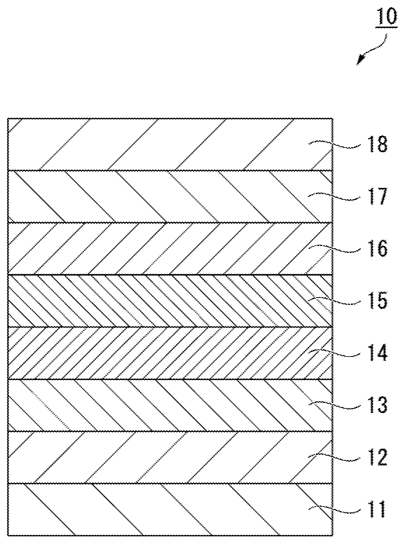

FIG. 1 is a schematic cross-sectional view showing one embodiment of a magnetic recording medium of the present invention.

A magnetic recording medium 10 of the present embodiment has a structure in which an adhesive layer 12, a soft magnetic layer 13, a first underlayer 14, a second underlayer 15, a magnetic layer 16, a protective layer 17, and a lubricating layer 18 are sequentially provided on a substrate 11.

### “Substrate”

As the substrate 11, for example, a non-magnetic substrate or the like in which a NiP or NiP alloy film is formed on a base made of metal or alloy material such as Al or an Al alloy can be used.

In addition, as the substrate 11, a non-magnetic substrate made of a non-metal material such as glass, ceramics, silicon, silicon carbide, carbon or a resin may be used, and a non-magnetic substrate in which a NiP or NiP alloy film is formed on a base made of this non-metal material may also be used.

### “Adhesive Layer”

The adhesive layer 12 prevents the progress of corrosion of the substrate 11 which may occur in a case where the substrate 11 and the soft magnetic layer 13, which is provided on the adhesive layer 12, are arranged in contact with each other.

The material of the adhesive layer 12 can be appropriately selected from, for example, Cr, a Cr alloy, Ti, a Ti alloy, CrTi, NiAl, and an AlRu alloy. The adhesive layer 12 can be formed by, for example, a sputtering method.

### “Soft Magnetic Layer”

The soft magnetic layer 13 preferably has a structure in which a first soft magnetic film, an interlayer made of a Ru film, and a second soft magnetic film are sequentially laminated. That is, the soft magnetic layer 13 preferably has a structure in which the interlayer made of a Ru film is sandwiched between the two soft magnetic films, whereby the soft magnetic films on and under the interlayer are antiferromagnetically coupled (AFC).

Examples of the material of the first soft magnetic film and the second soft magnetic film include a CoZrTa alloy and a CoFe alloy.

Any of Zr, Ta and Nb is preferably added to the CoFe alloy that is used for the first soft magnetic film and the second soft magnetic film. This accelerates the amorphization of the first soft magnetic film and the second soft magnetic film, makes it possible to improve the orientation of the first underlayer (seed layer) and makes it possible to reduce the flying height of a magnetic head.

The soft magnetic layer 13 can be formed by, for example, a sputtering method.

### “First Underlayer”

The first underlayer 14 is a layer for controlling the orientations and crystal sizes of the second underlayer 15 and the magnetic layer 16 that are provided on the first underlayer 14.

Examples of the first underlayer 14 include a Cr layer, a Ta layer, a Ru layer, a CrMo alloy layer, a CoW alloy layer, a CrW alloy layer, a CrV alloy layer, and a CrTi alloy layer.

The first underlayer 14 can be formed by, for example, a sputtering method.

### “Second Underlayer”

The second underlayer 15 is a layer that controls the orientation of the magnetic layer 16 to be favorable. The second underlayer 15 is preferably a Ru or Ru alloy layer.

The second underlayer 15 may be a single layer or may be composed of a plurality of layers. In a case where the second underlayer 15 is composed of a plurality of layers, all of the layers may be composed of the same material or at least one layer may be composed of a different material.

The second underlayer 15 can be formed by, for example, a sputtering method.

### “Magnetic Layer”

The magnetic layer 16 is made of a magnetic film in which the easy magnetization axis is directed in a perpendicular or parallel direction with respect to the substrate surface. The magnetic layer 16 is a layer containing Co and Pt and may be a layer further containing an oxide or Cr, B, Cu, Ta, Zr, or the like in order to improve SNR characteristics.

Examples of the oxide that is contained in the magnetic layer 16 include SiO₂, SiO, Cr₂O₃, CoO, Ta₂O₃, and TiO₂.

The magnetic layer 16 may be composed of a single layer or may be composed of a plurality of magnetic layers made of materials with different compositions.

For example, in a case where the magnetic layer 16 is composed of three layers of a first magnetic layer, a second magnetic layer, and a third magnetic layer sequentially laminated from below, the first magnetic layer preferably has a granular structure made of a material containing Co, Cr, and Pt and further containing an oxide. As the oxide that is contained in the first magnetic layer, for example, oxides of Cr, Si, Ta, Al, Ti, Mg, Co, or the like are preferably used. Among them, in particular, TiO₂, Cr₂O₃, SiO₂, and the like can be suitably used. In addition, the first magnetic layer is preferably made of a composite oxide to which two or more oxides have been added. Among them, in particular, Cr₂O₃—SiO₂, Cr₂O₃—TiO₂, SiO₂—TiO₂, and the like can be suitably used.

The first magnetic layer may contain, in addition to Co, Cr, Pt, and the oxide, one or more elements selected from B, Ta, Mo, Cu, Nd, W, Nb, Sm, Tb, Ru, and Re.

For the second magnetic layer, the same material as for the first magnetic layer can be used. The second magnetic layer preferably has a granular structure.

The third layer preferably has a non-granular structure made of a material containing Co, Cr, and Pt but containing no oxides. The third magnetic layer may contain, in addition to Co, Cr, and Pt, one or more elements selected from B, Ta, Mo, Cu, Nd, W, Nb, Sm, Tb, Ru, Re, and Mn.

In a case where the magnetic layer 16 is formed of a plurality of magnetic layers, a non-magnetic layer is preferably provided between the magnetic layers adjacent to each other. In a case where the magnetic layer 16 is made up of three layers of the first magnetic layer, the second magnetic layer and the third magnetic layer, it is preferable to provide a non-magnetic layer between the first magnetic layer and the second magnetic layer and a non-magnetic layer between the second magnetic layer and the third magnetic layer.

For the non-magnetic layer that is provided between the magnetic layers adjacent to each other in the magnetic layer 16, it is possible to suitably use, for example, Ru, a Ru alloy, a CoCr alloy, and a CoCrX1 alloy (X1 represents one or more elements selected from Pt, Ta, Zr, Re, Ru, Cu, Nb, Ni, Mn, Ge, Si, O, N, W, Mo, Ti, V, and B).

For the non-magnetic layer that is provided between the magnetic layers adjacent to each other in the magnetic layer 16, an alloy material containing an oxide, a metallic nitride or a metallic carbide is preferably used. Specifically, as the oxide, for example, SiO₂, Al₂O₃, Ta₂O₅, Cr₂O₃, MgO, Y₂O₃, TiO₂ and the like can be used. As the metallic nitride, for example, AlN, Si₃N₄, TaN, CrN and the like can be used. As the metallic carbide, for example, TaC, BC, SiC and the like can be used.

The non-magnetic layer can be formed by, for example, a sputtering method.

The magnetic layer 16 is preferably a magnetic layer for perpendicular magnetic recording in which the easy magnetization axis is directed in a direction perpendicular to the substrate surface in order to realize a higher recording density. The magnetic layer 16 may be a magnetic layer for in-plane magnetic recording.

The magnetic layer 16 may be formed by any well-known conventional method such as a deposition method, an ion beam sputtering method, or a magnetron sputtering method. The magnetic layer 16 is normally formed by a sputtering method.

### “Protective Layer”

The protective layer 17 protects the magnetic layer 16. The protective layer 17 may be composed of a single layer or may be composed of a plurality of layers. As the material of the protective layer 17, carbon, nitrogen-containing carbon, silicon carbide, and the like can be exemplified.

As the protective layer 17, a carbon-based protective layer can be preferably used, and, in particular, an amorphous carbon protective layer is preferable. When the protective layer 17 is a carbon-based protective layer, an interaction with the hydroxyl group contained in the fluorine-containing ether compound in the lubricating layer 18 is further enhanced, which is preferable.

The adhesive force between the carbon-based protective layer and the lubricating layer 18 can be controlled by forming the carbon-based protective layer with hydrogenated carbon and/or nitrogenated carbon and adjusting the hydrogen content and/or the nitrogen content of the carbon-based protective layer. The hydrogen content in the carbon-based protective layer is preferably 3 to 20 atomic % when measured by the hydrogen forward scattering method (HFS). In addition, the nitrogen content of the carbon-based protective layer is preferably 4 to 15 atomic % when measured by X-ray photoelectron spectroscopy (XPS).

The hydrogen and/or nitrogen that are contained in the carbon-based protective layer do not need to be uniformly contained throughout the entire carbon-based protective layer. The carbon-based protective layer is suitably formed as, for example, a composition gradient layer in which nitrogen is contained in the lubricating layer 18 side of the protective layer 17 and hydrogen is contained in the magnetic layer 16 side of the protective layer 17. In this case, the adhesive force between the magnetic layer 16 and the carbon-based protective layer and the adhesive force between the lubricating layer 18 and the carbon-based protective layer further improve.

The film thickness of the protective layer 17 is preferably set to 1 nm to 7 nm. When the film thickness of the protective layer 17 is 1 nm or more, performance as the protective layer 17 can be sufficiently obtained. The film thickness of the protective layer 17 is preferably 7 nm or less from the viewpoint of reducing the thickness of the protective layer 17.

As a method for forming the protective layer 17, it is possible to use a sputtering method in which a carbon-containing target material is used, a chemical vapor deposition (CVD) method in which a hydrocarbon raw material such as ethylene or toluene is used, an ion beam deposition (IBD) method, and the like.

In the case of forming a carbon-based protective layer as the protective layer 17, the carbon-based protective layer can be formed by, for example, a DC magnetron sputtering method. Particularly, in the case of forming a carbon-based protective layer as the protective layer 17, an amorphous carbon protective layer is preferably formed by a plasma CVD method. The amorphous carbon protective layer formed by the plasma CVD method has a uniform surface with small roughness.

### “Lubricating Layer”

The lubricating layer 18 prevents contamination of the magnetic recording medium 10. In addition, the lubricating layer 18 reduces frictional force of a magnetic head of a magnetic recording/reproducing device, which slides on the magnetic recording medium 10, thereby improving the durability of the magnetic recording medium 10.

The lubricating layer 18 is formed in contact with the protective layer 17 as shown in FIG. 1. The lubricating layer 18 contains the above-described fluorine-containing ether compound.

In a case where the protective layer 17, which is placed below the lubricating layer 18, is a carbon-based protective layer, particularly, the lubricating layer 18 is bound to the protective layer 17 with a high binding force. As a result, the magnetic recording medium 10 in which the surface of the protective layer 17 is coated with the lubricating layer 18 at a high coating rate in spite of a thin thickness is likely to be obtained, and contamination on the surface of the magnetic recording medium 10 can be effectively prevented.

The average film thickness of the lubricating layer 18 is preferably 0.5 nm (5 Å) to 2.0 nm (20 Å) and more preferably 0.5 nm (5 Å) to 1.0 nm (10 Å). When the average film thickness of the lubricating layer 18 is 0.5 nm or more, the lubricating layer 18 does not have an island shape or a mesh shape and is formed in a uniform film thickness. For this reason, the surface of the protective layer 17 can be coated with the lubricating layer 18 at a high coating rate. In addition, when the average film thickness of the lubricating layer 18 is set to 2.0 nm or less, it is possible to sufficiently reduce the thickness of the lubricating layer 18 and to sufficiently decrease the flying height of a magnetic head.

In a case where the surface of the protective layer 17 is not sufficiently coated with the lubricating layer 18 at a high coating rate, an environmental substance adsorbed to the surface of the magnetic recording medium 10 passes through voids in the lubricating layer 18 and intrudes below the lubricating layer 18. The environmental substance that has intruded below the lubricating layer 18 is adsorbed and bound to the protective layer 17 and generates a contamination substance. At the time of reproducing magnetic records, the generated contamination substance (aggregated component) adheres (transfers) to a magnetic head as a smear to break the magnetic head or degrade the magnetic recording/reproducing characteristics of magnetic recording/reproducing devices.

Examples of the environmental substance that generates the contamination substance include siloxane compounds (cyclic siloxane and linear siloxane), ionic impurities, hydrocarbons having a relatively high molecular weight such as octacosane, and plasticizers such as dioctyl phthalate. Examples of metal ions contained in the ionic impurities include a sodium ion and a potassium ion. Examples of inorganic ions contained in the ionic impurities include a chlorine ion, a bromine ion, a nitrate ion, a sulfate ion, and an ammonium ion. Examples of organic ions contained in the ionic impurities include an oxalate ion and a formate ion.

### “Method for Forming Lubricating Layer”

Examples of methods for forming the lubricating layer 18 include a method in which a magnetic recording medium that is not yet fully manufactured and thus includes the individual layers up to the protective layer 17 formed on the substrate 11 is prepared and a solution for forming a lubricating layer is applied onto the protective layer 17 and dried.

The solution for forming a lubricating layer can be obtained by dispersing and dissolving the above-described lubricant for a magnetic recording medium of the embodiment in a solvent as necessary and adjusting the viscosity and concentration to be suitable for application methods.

Examples of solvents used for the solution for forming a lubricating layer include fluorine-based solvents such as VERTREL (registered trademark) XF (trade name, manufactured by Dupont-Mitsui Fluorochemicals Co., Ltd.).

A method for applying the solution for forming a lubricating layer is not particularly limited, and examples thereof include a spin coating method, a spraying method, a paper coating method, and a dipping method.

In a case of using a dipping method, it is possible to use, for example, a method shown below. First, the substrate 11 on which the individual layers up to the protective layer 17 have been formed is immersed in the solution for forming a lubricating layer that has been placed in an immersion vessel of a dip coater. Next, the substrate 11 is lifted from the immersion vessel at a predetermined speed. As a result, the solution for forming a lubricating layer is applied to the surface of the protective layer 17 on the substrate 11.

The use of the dipping method makes it possible to uniformly apply the solution for forming a lubricating layer to the surface of the protective layer 17 and makes it possible to form the lubricating layer 18 on the protective layer 17 in a uniform film thickness.

In the present embodiment, a burnishing (precision polishing) step is preferably performed after the lubricating layer 18 is formed on the surface of the substrate 11. By performing the burnishing step, projection defects and particles present on the surface of the substrate 11 on which the lubricating layer 18 has been formed can be removed, and the magnetic recording medium 10 with a smooth surface can be obtained. If the surface of the magnetic recording medium 10 is smooth, the spacing loss between the magnetic recording medium 10 and a magnetic head can be reduced and the signal characteristics can improve.

As the burnishing step, for example, a step of scanning burnishing tape on the surface of the substrate 11 on which the lubricating layer 18 has been formed can be performed. As the burnishing tape, one made of a resin film holding abrasive grains can be used. The grain size of the abrasive grains can be set to, for example, #6000 to #20000.

In the present embodiment, a heat treatment is preferably carried out on the substrate 11 on which the lubricating layer 18 has been formed. The heat treatment improves the adhesion properties between the lubricating layer 18 and the protective layer 17 and improves the adhesive force between the lubricating layer 18 and the protective layer 17.

The heat treatment temperature is preferably set to 100° C. to 180° C. When the heat treatment temperature is 100° C. or higher, an effect on improvement in the adhesion properties between the lubricating layer 18 and the protective layer 17 can be sufficiently obtained. In addition, when the heat treatment temperature is set to 180° C. or lower, it is possible to prevent thermal decomposition of the lubricating layer 18. The heat treatment time is preferably set to 10 to 120 minutes.

The magnetic recording medium 10 of the present embodiment includes at least the magnetic layer 16, the protective layer 17, and the lubricating layer 18 sequentially provided on the substrate 11. In the magnetic recording medium 10 of the present embodiment, the lubricating layer 18 containing the above-described fluorine-containing ether compound is formed in contact with the protective layer 17. This lubricating layer 18 is highly effective in suppressing corrosion of the magnetic recording medium 10. For this reason, the magnetic recording medium 10 of the present embodiment has less contamination substances present on the surface, an excellent corrosion resistance, and favorable reliability and durability. In addition, since the magnetic recording medium 10 of the present embodiment has the lubricating layer 18 highly effective in suppressing corrosion, the thickness of the protective layer 17 and/or the lubricating layer 18 can be reduced. In addition, in the lubricating layer 18 in the magnetic recording medium 10 of the present embodiment, foreign matter (smears) are less likely to be generated, and pickup can be suppressed.

### EXAMPLES

Hereinafter, the present invention will be described more specifically with reference to examples and comparative examples. The present invention is not limited to the following examples.

### Example 1

A compound represented by Formula (A) above was produced through a method shown below.

### (First Reaction)

9.4 g (20 mmol) of a compound represented by HOCH₂CF₂O(CF₂CF₂O)ᵣCF₂CH₂OH (r indicating the average degree of polymerization in the formula is 2.5) (number average molecular weight: 468, molecular weight distribution: 1.1), 1.76 g (44 mmol) of 60% sodium hydride, and 15.6 mL of N,N-dimethylformamide were added to a 200 mL eggplant flask in a nitrogen gas atmosphere and stirred until the mixture became uniform at room temperature. 6.34 g (42 mmol) of 2-(2-bromoethyl)oxirane was added to this uniform liquid and reacted by being stirred at 40° C. for 2 hours.

A reaction product obtained after the reaction was cooled to 25° C., 80 mL of water was added thereto to stop the reaction, the mixture was moved to a separatory funnel, and extraction was performed twice with 150 mL of ethyl acetate. An organic layer thereof was washed with saturated saline and dehydrated with anhydrous sodium sulfate. After the drying agent was filtered, the filtrate was concentrated, and the residue was purified by silica gel column chromatography, thereby obtaining 3.7 g (molecular weight: 608, 6.0 mmol) of a compound represented by Formula (10) below as an intermediate compound 1.

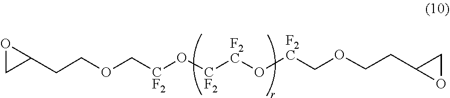

(In Formula (10), r indicating the average degree of polymerization is 2.5.)

### (Second Reaction)

14.0 g of the compound represented by HOCH₂CF₂O(CF₂CF₂O)ᵣCF₂CH₂OH (r indicating the average degree of polymerization in the formula is 2.5) (number average molecular weight: 468, molecular weight distribution: 1.1), 3.89 g (molecular weight: 216.28, 18 mmol) of a compound represented by Formula (11) below, and 28 mL of t-butanol were added to a 200 mL eggplant flask in a nitrogen gas atmosphere and stirred until the mixture became uniform at room temperature. 1.0 g (molecular weight: 112.2, 9 mmol) of potassium tert-butoxide was further added to this uniform solution and reacted by being stirred at 70° C. for 16 hours.

The compound represented by Formula (11) was synthesized by protecting one hydroxyl group of 1,3-propanediol with a tetrahydropyranyl group (THP group) and reacting epibromohydrin with the other hydroxyl group.

A reaction product obtained after the reaction was cooled to 25° C., moved to a separatory funnel containing 100 mL of water, and extracted three times with 100 mL of ethyl acetate. An organic layer thereof was washed with water and dehydrated with anhydrous sodium sulfate. After the drying agent was filtered, the filtrate was concentrated, and the residue was purified by silica gel column chromatography, thereby obtaining 8.2 g (molecular weight: 684, 12.0 mmol) of a compound represented by Formula (12) below as an intermediate compound 2.

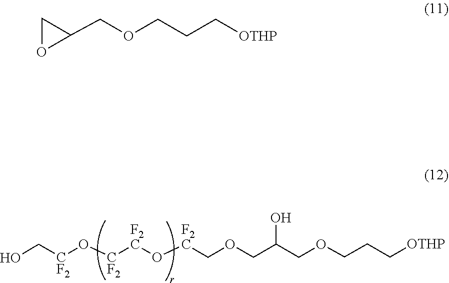

(In Formula (11), THP represents a tetrahydropyranyl group.)

(In Formula (12), r indicating the average degree of polymerization is 2.5.)

### (Third Reaction)

6.8 g of the intermediate compound 2 represented by Formula (12) (r indicating the average degree of polymerization in the formula is 2.5), 0.34 g of potassium tert-butoxide, and 9.4 mL of t-butanol were added to a 200 mL eggplant flask in a nitrogen gas atmosphere and stirred until the mixture became uniform at room temperature. 1.8 g of the intermediate compound 1 represented by Formula (10) (r indicating the average degree of polymerization in the formula is 2.5) was further added to this uniform liquid and reacted by being stirred at 70° C. for 16 hours.

A reaction product obtained after the reaction was cooled to 25° C., moved to a separatory funnel containing 100 ml of water, and extracted three times with 100 mL of ethyl acetate. An organic layer thereof was washed with water and dehydrated with anhydrous sodium sulfate. After the drying agent was filtered, the filtrate was concentrated, and the residue was purified by silica gel column chromatography, thereby obtaining 3.3 g (molecular weight: 1809, 1.8 mmol) of the compound represented by Formula (A) above (Fpa₁ and Fpa₂ in Formula (A) are represented by Formula (AF), ra indicating the average degree of polymerization in Fpa₁ is 2.5, and ra indicating the average degree of polymerization in Fpa₂ is 2.5).

¹H-NMR and ¹⁹F-NMR measurements of the obtained compound (A) were carried out, and the structure was identified from the following results.

¹H-NMR (CD₃COCD₃): δ [ppm]=1.38 to 1.75 (8H), 3.37 to 4.31 (46H)

¹⁹F-NMR (CD₃COCD₃): δ [ppm]=−78.6 (6F), −81.3 (6F), −90.0 to −88.5 (30F)

### Example 2

The same operation as in Example 1 was carried out except that 13.9 g of a compound represented by HOCH₂CF₂CF₂O(CF₂CF₂CF₂O)_{q}CF₂CF₂CH₂OH (q indicating the average degree of polymerization in the formula is 2.5) (number average molecular weight: 693, molecular weight distribution: 1.1) was used instead of the compound represented by HOCH₂CF₂O(CF₂CF₂O)ᵣCF₂CH₂OH (r indicating the average degree of polymerization in the formula was 2.5) in the first reaction, and that 20.8 g of a compound represented by HOCH₂CF₂CF₂O(CF₂CF₂CF₂O)_{q}CF₂CF₂CH₂OH (q indicating the average degree of polymerization in the formula is 2.5) (number average molecular weight: 693, molecular weight distribution: 1.1) was used instead of the compound represented by HOCH₂CF₂O(CF₂CF₂O)ᵣCF₂CH₂OH (r indicating the average degree of polymerization in the formula was 2.5) and 6.02 g of a compound represented by Formula (13) below was used instead of the compound represented by Formula (11) in the second reaction, thereby obtaining 4.7 g (molecular weight: 2632, 1.8 mmol) of the compound represented by Formula (B) above (Fdb₁ and Fdb₂ in Formula (B) are represented by Formula (BF), qb indicating the average degree of polymerization in Fdb₁ is 2.5, and qb indicating the average degree of polymerization in Fdb₂ is 2.5).

The compound represented by Formula (13) was synthesized by the following method.

A tert-butyldimethylsilyl group (TBS group) was introduced into a primary hydroxyl group in 3-allyloxy-1,2-propanediol, and a methoxymethyl group (MOM group) was introduced into a secondary hydroxyl group in the obtained compound. After the TBS group in the obtained compound was removed, 2-(chloropropoxy)tetrahydro-2H-pyran was reacted with the generated primary hydroxyl group. The double bond of the obtained compound was oxidized. The compound represented by Formula (13) was obtained through the above-described steps.

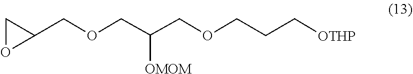

(In Formula (13), THP represents a tetrahydropyranyl group, and MOM represents a methoxymethyl group.)

¹H-NMR and ¹⁹F-NMR measurements of the obtained compound (B) were carried out, and the structure was identified from the following results.

¹H-NMR (CD₃COCD₃): δ [ppm]=1.37 to 1.81 (8H), 3.36 to 4.35 (58H)

¹⁹F-NMR (acetone-D₆): δ [ppm]=−84.0 to −83.0 (30F), −86.4 (12F), −124.3 (12F), −130.0 to −129.0 (15F)

### Example 3

The same operation as in Example 1 was carried out except that 12.7 g of the compound represented by HOCH₂CF₂O(CF₂CF₂O)ₛ(CF₂O)ₜCF₂CH₂OH (s and t indicating the average degree of polymerization in the formula are 2.5) (number average molecular weight: 633, molecular weight distribution: 1.1) was used instead of the compound represented by HOCH₂CF₂O(CF₂CF₂O)ᵣCF₂CH₂OH (r indicating the average degree of polymerization in the formula is 2.5) in the first reaction, and that 19.0 g of the compound represented by HOCH₂CF₂O(CF₂CF₂O)ₛ(CF₂O)ₜCF₂CH₂OH (s and t indicating the average degree of polymerization in the formula are 2.5) (number average molecular weight: 633, molecular weight distribution: 1.1) was used instead of the compound represented by HOCH₂CF₂O(CF₂CF₂O)ᵣCF₂CH₂OH (r indicating the average degree of polymerization in the formula is 2.5) and 3.10 g of a compound represented by Formula (14) below was used instead of the compound represented by Formula (11) in the second reaction, thereby obtaining 4.0 g (molecular weight: 2216, 1.8 mmol) of the compound represented by Formula (C) above (Ffc₁ and Ffc₂ in Formula (C) are represented by Formula (CF), sc and tc indicating the average degree of polymerization in Ffc₁ are 2.5, and sc and tc indicating the average degree of polymerization in Ffc₂ are 2.5).

The compound represented by Formula (14) was synthesized by introducing a tetrahydropyranyl group (THP group) into a primary hydroxyl group in 3-buten-1-ol and oxidizing the double bond of the obtained compound.

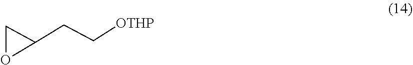

(In Formula (14), THP represents a tetrahydropyranyl group.)

¹H-NMR and ¹⁹F-NMR measurements of the obtained compound (C) were carried out, and the structure was identified from the following results.

¹H-NMR (CD₃COCD₃): δ [ppm]=1.34 to 1.67 (8H), 3.39 to 4.34 (38H)

¹⁹F-NMR (CD₃COCD₃): δ [ppm]=−55.6 to −50.6 (15F), −77.7 (6F), −80.3 (6F), −91.0 to −88.5 (30F)

### Example 4

The same operation as in Example 1 was carried out except that 4.76 g of a compound represented by Formula (15) below was used instead of the compound represented by Formula (11) in the second reaction, thereby obtaining 3.4 g (molecular weight: 1897, 1.8 mmol) of the compound represented by Formula (D) above (Fpd₁ and Fpd₂ in Formula (D) are represented by Formula (DF), rd indicating the average degree of polymerization in Fpd₁ is 2.5, and rd indicating the average degree of polymerization in Fpd₂ is 2.5).

The compound represented by Formula (15) was synthesized by the following method.

1,2,4-Butanetriol was reacted with benzaldehyde dimethylacetal to synthesize a compound in which hydroxyl groups bound to 2- and 4-position carbons of 1,2,4-butanetriol were protected. This compound was reacted with 2-bromoethyloxirane to synthesize the compound represented by Formula (15).

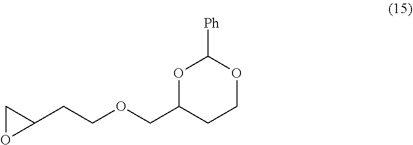 [SMILES: c1ccc(C2OCCC(COCCC3CO3)O2)cc1]

(In Formula (15), Ph represents a phenyl group.)

¹H-NMR and ¹⁹F-NMR measurements of the obtained compound (D) were carried out, and the structure was identified from the following results.

¹H-NMR (CD₃COCD₃): δ [ppm]=1.34 to 1.65 (12H), 3.42 to 4.35 (50H)

¹⁹F-NMR (CD₃COCD₃): δ [ppm]=−78.6 (6F), −81.3 (6F), −90.0 to −88.5 (30F)

### Example 5

The same operation as in Example 2 was carried out except that 3.64 g of a compound represented by Formula (16) below was used instead of the compound represented by Formula (13) in the second reaction, thereby obtaining 4.4 g (molecular weight: 2456, 1.8 mmol) of the compound represented by Formula (E) above (Fde₁ and Fde₂ in Formula (E) are represented by Formula (EF), qe indicating the average degree of polymerization in Fde₁ is 2.5, and qe indicating the average degree of polymerization in Fde₂ is 2.5).

The compound represented by Formula (16) was synthesized by oxidizing a compound obtained by protecting ethylene glycol monoallyl ether using dihydropyran.

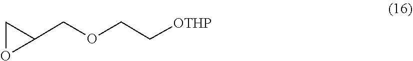

(In Formula (16), THP represents a tetrahydropyranyl group.)

¹H-NMR and ¹⁹F-NMR measurements of the obtained compound (E) were carried out, and the structure was identified from the following results.

¹H-NMR (CD₃COCD₃): δ [ppm]=1.37 to 1.64 (4H), 3.42 to 4.35 (46H) ¹⁹F-NMR (acetone-D₆): δ [ppm]=−84.0 to −83.0 (30F), −86.4 (12F), −124.3 (12F), −130.0 to −129.0 (15F)

### Example 6

The same operation as in Example 2 was carried out except that 3.89 g of a compound represented by Formula (17) below was used instead of the compound represented by Formula (13) in the second reaction, thereby obtaining 4.5 g (molecular weight: 2484, 1.8 mmol) of the compound represented by Formula (F) above (Fdf₁ and Fdf₂ in Formula (F) are represented by Formula (FF), qf indicating the average degree of polymerization in Fdf₁ is 2.5, and qf indicating the average degree of polymerization in Fdf₂ is 2.5).

The compound represented by Formula (17) was synthesized by oxidizing the double bond of a compound obtained by reacting 3-buten-1-ol with 2-bromoethoxytetrahydropyran.

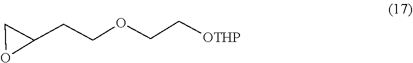

(In Formula (17), THP represents a tetrahydropyranyl group.)

¹H-NMR and ¹⁹F-NMR measurements of the obtained compound (F) were carried out, and the structure was identified from the following results.

¹H-NMR (CD₃COCD₃): δ [ppm]=1.34 to 1.59 (8H), 3.37 to 4.38 (46H)

¹⁹F-NMR (acetone-D₆): δ [ppm]=−84.0 to −83.0 (30F), −86.4 (12F), −124.3 (12F), −130.0 to −129.0 (15F)

### Example 7

The same operation as in Example 3 was carried out except that 4.51 g of a compound represented by Formula (18) below was used instead of the compound represented by Formula (14) in the second reaction, thereby obtaining 4.3 g (molecular weight: 2364, 1.8 mmol) of the compound represented by Formula (G) above (Ffg₁ and Ffg₂ in Formula (G) are represented by Formula (GF), sg and tg indicating the average degree of polymerization in Ffg₁ are 2.5, and sg and tg indicating the average degree of polymerization in Ffg₂ are 2.5).

The compound represented by Formula (18) was synthesized by the following method.

1,2,4-Butanetriol was reacted with benzaldehyde dimethylacetal to synthesize a compound in which hydroxyl groups bound to 2- and 4-position carbons of 1,2,4-butanetriol were protected. This compound was reacted with epibromohydrin to synthesize the compound represented by Formula (18).

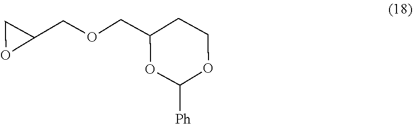 [SMILES: c1ccc(C2OCCC(COCC3CO3)O2)cc1]

(In Formula (18), Ph represents a phenyl group.)

¹H-NMR and ¹⁹F-NMR measurements of the obtained compound (G) were carried out, and the structure was identified from the following results.

¹H-NMR (CD₃COCD₃): δ [ppm]=1.34 to 1.67 (8H), 3.38 to 4.33 (50H)

¹⁹F-NMR (CD₃COCD₃): δ [ppm]=−55.6 to −50.6 (15F), −77.7 (6F), −80.3 (6F), −91.0 to −88.5 (30F)

### Example 8

The same operation as in Example 1 was carried out except that 2-(3-bromopropyl)oxirane was used instead of 2-(2-bromoethyl)oxirane in the first reaction and 3.35 g of a compound represented by Formula (19) below was used instead of the compound represented by Formula (11) in the second reaction, thereby obtaining 3.2 g (molecular weight: 1777, 1.8 mmol) of the compound represented by Formula (H) above (Fph₁ and Fph₂ in Formula (H) are represented by Formula (HF), rh indicating the average degree of polymerization in Fph₁ is 2.5, and rh indicating the average degree of polymerization in Fph₂ is 2.5).

The compound represented by Formula (19) was synthesized by introducing a tetrahydropyranyl group (THP group) into a primary hydroxyl group in 4-penten-1-ol and oxidizing the double bond of the obtained compound.

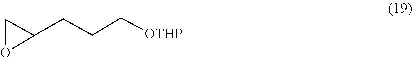

(In Formula (19), THP represents a tetrahydropyranyl group.)

¹H-NMR and ¹⁹F-NMR measurements of the obtained compound (H) were carried out, and the structure was identified from the following results.

¹H-NMR (CD₃COCD₃): δ [ppm]=1.34 to 1.65 (16H), 3.42 to 4.35 (38H)

¹⁹F-NMR (CD₃COCD₃): δ [ppm]=−78.6 (6F), −81.3 (6F), −90.0 to −88.5 (30F)

### Example 9

The same operation as in Example 2 was carried out except that 2-(3-bromopropyl)oxirane was used instead of 2-(2-bromoethyl)oxirane in the first reaction and 6.99 g of a compound represented by Formula (20) below was used instead of the compound represented by Formula (13) in the second reaction, thereby obtaining 4.8 g (molecular weight: 2688, 1.8 mmol) of the compound represented by Formula (I) above (Fdi₁ and Fdi₂ in Formula (I) are represented by Formula (IF), qi indicating the average degree of polymerization in Fdi₁ is 2.5, and qi indicating the average degree of polymerization in Fdi₂ is 2.5).

The compound represented by Formula (20) was synthesized by the following method.

A first compound was obtained by oxidizing a compound in which ethylene glycol monoallyl ether was protected using dihydropyran. The first compound was reacted with a hydroxyl group of 4-penten-1-ol to obtain a second compound. The double bond of a compound in which a secondary hydroxyl group of the second compound was protected with a THP group was oxidized. The compound represented by Formula (20) was obtained through the above-described steps.

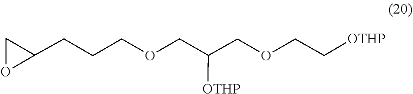

(In Formula (20), THP represents a tetrahydropyranyl group.)

¹H-NMR and ¹⁹F-NMR measurements of the obtained compound (I) were carried out, and the structure was identified from the following results.

¹H-NMR (CD₃COCD₃): δ [ppm]=1.38 to 1.58 (16H), 3.38 to 4.41 (58H)

¹⁹F-NMR (acetone-D₆): δ [ppm]=−84.0 to −83.0 (30F), −86.4 (12F), −124.3 (12F), −130.0 to −129.0 (15F)

### Example 10

The same operation as in Example 3 was carried out except that 2-(3-bromopropyl)oxirane was used instead of 2-(2-bromoethyl)oxirane in the first reaction and 5.77 g of a compound represented by Formula (21) below was used instead of the compound represented by Formula (14) in the second reaction, thereby obtaining 4.4 g (molecular weight: 2452, 1.8 mmol) of the compound represented by Formula (J) above (Ffj₁ and Ffj₂ in Formula (J) are represented by Formula (JF), sj and tj indicating the average degree of polymerization in Ffj₁ are 2.5, and sj and tj indicating the average degree of polymerization in Ffj₂ are 2.5).

The compound represented by Formula (21) was synthesized by the following method.

A tert-butyldimethylsilyl (TBS) group was introduced as a protective group into a primary hydroxyl group in 3-allyloxy-1,2-propanediol, and a methoxymethyl (MOM) group was introduced as a protective group into a secondary hydroxyl group in the obtained compound. After that, the TBS group was removed from the compound, and 2-bromoethoxytetrahydropyran was reacted with the generated primary hydroxyl group. The double bond of the obtained compound was oxidized. The compound represented by Formula (21) was obtained through the above-described steps.

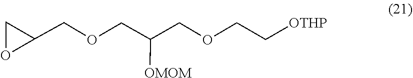

(In Formula (21), THP represents a tetrahydropyranyl group, and MOM represents a methoxymethyl group.)

¹H-NMR and ¹⁹F-NMR measurements of the obtained compound (J) were carried out, and the structure was identified from the following results.

¹H-NMR (CD₃COCD₃): δ [ppm]=1.34 to 1.66 (8H), 3.39 to 4.35 (58H)

¹⁹F-NMR (CD₃COCD₃): δ [ppm]=−55.6 to −50.6 (15F), −77.7 (6F), −80.3 (6F), −91.0 to −88.5 (30F)

### Example 11

The same operation as in Example 1 was carried out except that 20.8 g of the compound represented by HOCH₂CF₂CF₂O(CF₂CF₂CF₂O)_{q}CF₂CF₂CH₂OH (q indicating the average degree of polymerization in the formula is 2.5) (number average molecular weight: 693, molecular weight distribution: 1.1) was used instead of the compound represented by HOCH₂CF₂O(CF₂CF₂O)ᵣCF₂CH₂OH (r indicating the average degree of polymerization in the formula is 2.5) and 4.51 g of the compound represented by Formula (18) above was used instead of the compound represented by Formula (11) in the second reaction, thereby obtaining 4.2 g (molecular weight: 2319, 1.8 mmol) of the compound represented by Formula (K) above (Fdk₁ and Fpk₁ in Formula (K) are represented by Formula (KF), qk indicating the average degree of polymerization in Fdk₁ is 2.5, and rk indicating the average degree of polymerization in Fpk₁ is 2.5).

¹H-NMR and ¹⁹F-NMR measurements of the obtained compound (K) were carried out, and the structure was identified from the following results.

¹H-NMR (CD₃COCD₃): δ [ppm]=1.34 to 1.63 (8H), 3.39 to 4.35 (50H)

¹⁹F-NMR (CD₃COCD₃): δ [ppm]=−78.6 (2F), −81.3 (2F), −84.0 to −83.0 (20F), −86.4 (8F), −90.0 to −88.5 (10F), −124.3 (8F), −130.0 to −129.0 (10F)

### Example 12

The same operation as in Example 1 was carried out except that the compound represented by HOCH₂CF₂O(CF₂CF₂O)ₛ(CF₂O)ₜCF₂CH₂OH (s and t indicating the average degree of polymerization in the formula are 2.5) (number average molecular weight: 633, molecular weight distribution: 1.1) was used instead of the compound represented by HOCH₂CF₂O(CF₂CF₂O)ᵣCF₂CH₂OH (r indicating the average degree of polymerization in the formula is 2.5) and 6.02 g of a compound represented by Formula (22) below was used instead of the compound represented by Formula (11) in the second reaction, thereby obtaining 4.1 g (molecular weight: 2287, 1.8 mmol) of the compound represented by Formula (L) above (Ffl₁ and Fpl₁ in Formula (L) are represented by Formula (LF), sl and tl indicating the average degree of polymerization in Ffl₁ are 2.5, and rl indicating the average degree of polymerization in Fpl₁ is 2.5).

The compound represented by Formula (22) was synthesized by the following method.

A first compound was obtained by protecting ethylene glycol monoallyl ether using dihydropyran and oxidizing the resulting compound. Next, the first compound was reacted with a hydroxyl group of 3-buten-1-ol to obtain a second compound. The compound represented by Formula (22) was obtained by protecting a secondary hydroxyl group of the obtained second compound with a MOM group and oxidizing the double bond.

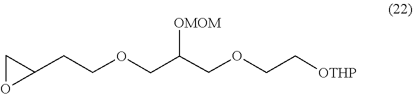

(In Formula (22), THP represents a tetrahydropyranyl group, and MOM represents a methoxymethyl group.)

¹H-NMR and ¹⁹F-NMR measurements of the obtained compound (L) were carried out, and the structure was identified from the following results.

¹H-NMR (CD₃COCD₃): δ [ppm]=1.35 to 1.62 (8H), 3.41 to 4.35 (58H)

¹⁹F-NMR (CD₃COCD₃): δ [ppm]=−55.6 to −50.6 (10F), −77.7 (4F), −78.6 (2F), −80.3 (4F), −81.3 (2F), −90.0 to −88.5 (30F)

### Example 13

The same operation as in Example 8 was carried out except that 20.8 g of the compound represented by HOCH₂CF₂CF₂O(CF₂CF₂CF₂O)_{q}CF₂CF₂CH₂OH (q indicating the average degree of polymerization in the formula is 2.5) (number average molecular weight: 693, molecular weight distribution: 1.1) was used instead of the compound represented by HOCH₂CF₂O(CF₂CF₂O)ᵣCF₂CH₂OH (r indicating the average degree of polymerization in the formula is 2.5) and 3.89 g of the compound represented by

Formula (11) above was used instead of the compound represented by Formula (19) in the second reaction, thereby obtaining 4.1 g (molecular weight: 2287, 1.8 mmol) of the compound represented by Formula (M) above (Fdm₁ and Fpm₁ in Formula (M) are represented by Formula (MF), qm indicating the average degree of polymerization in Fdm₁ is 2.5, and rm indicating the average degree of polymerization in Fpm₁ is 2.5).

¹H-NMR and ¹⁹F-NMR measurements of the obtained compound (M) were carried out, and the structure was identified from the following results.

¹H-NMR (CD₃COCD₃): δ [ppm]=1.35 to 1.56 (12H), 3.41 to 4.37 (46H)

¹⁹F-NMR (CD₃COCD₃): δ [ppm]=−78.6 (2F), −81.3 (2F), −84.0 to −83.0 (20F), −86.4 (8F), −90.0 to −88.5 (10F), −124.3 (8F), −130.0 to −129.0 (10F)

### Example 14

The same operation as in Example 2 was carried out except that 14.0 g of the compound represented by HOCH₂CF₂O(CF₂CF₂O)ᵣCF₂CH₂OH (r indicating the average degree of polymerization in the formula is 2.5) (number average molecular weight: 468, molecular weight distribution: 1.1) was used instead of the compound represented by HOCH₂CF₂CF₂O(CF₂CF₂CF₂O)_{q}CF₂CF₂CH₂OH (q indicating the average degree of polymerization in the formula is 2.5) in the second reaction, thereby obtaining 3.9 g (molecular weight: 2182, 1.8 mmol) of the compound represented by Formula (N) above (Fdn₁ and Fpn₁ in Formula (N) are represented by Formula (NF), qn indicating the average degree of polymerization in Fdn₁ is 2.5, and rn indicating the average degree of polymerization in Fpn₁ is 2.5).

¹H-NMR and ¹⁹F-NMR measurements of the obtained compound (N) were carried out, and the structure was identified from the following results.

¹H-NMR (CD₃COCD₃): δ [ppm]=1.39 to 1.61 (8H), 3.41 to 4.45 (58H)

¹⁹F-NMR (acetone-d₆): δ [ppm]=−78.6 (4F), −81.3 (4F), −84.0 to −83.0 (10F), −86.4 (4F), −90.0 to −88.5 (24F), −124.3 (4F), −130.0 to −129.0 (5F)

### Example 15

The same operation as in Example 14 was carried out except that 4.76 g of the compound represented by Formula (15) above was used instead of the compound represented by Formula (13) in the second reaction, thereby obtaining 3.8 g (molecular weight: 2122, 1.8 mmol) of the compound represented by Formula (O) above (Fdo₁ and Fpo₁ in Formula (O) are represented by Formula (OF), qo indicating the average degree of polymerization in Fdo₁ is 2.5, and ro indicating the average degree of polymerization in Fpo₁ is 2.5).

¹H-NMR and ¹⁹F-NMR measurements of the obtained compound (O) were carried out, and the structure was identified from the following results.

¹H-NMR (CD₃COCD₃): δ [ppm]=1.34 to 1.58 (12H), 3.38 to 4.41 (50H)

¹⁹F-NMR (acetone-d₆): δ [ppm]=−78.6 (4F), −81.3 (4F), −84.0 to −83.0 (10F), −86.4 (4F), −90.0 to −88.5 (24F), −124.3 (4F), −130.0 to −129.0 (5F)

### Example 16

The same operation as in Example 2 was carried out except that a compound represented by HOCH₂CF₂O(CF₂CF₂O)ₛ(CF₂O)ₜCF₂CH₂OH (s and t indicating the average degree of polymerization in the formula are 2.5) (number average molecular weight: 633, molecular weight distribution: 1.1) was used instead of the compound represented by HOCH₂CF₂CF₂O(CF₂CF₂CF₂O)_{q}CF₂CF₂CH₂OH (q indicating the average degree of polymerization in the formula is 2.5) and 4.15 g of a compound represented by Formula (23) below was used instead of the compound represented by Formula (13) in the second reaction, thereby obtaining 4.3 g (molecular weight: 2392, 1.8 mmol) of the compound represented by Formula (P) above (Fdp₁ and Ffp₁ in Formula (P) are represented by Formula (PF), qp indicating the average degree of polymerization in Fdp₁ is 2.5, and sp and tp indicating the average degree of polymerization in Ffp₁ are 2.5).

The compound represented by Formula (23) was synthesized by protecting one hydroxyl group of 1,4-butanediol with a THP group and reacting epibromohydrin with the other hydroxyl group.

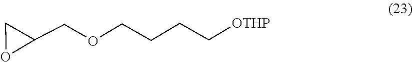

(In Formula (23), THP represents a tetrahydropyranyl group.)

¹H-NMR and ¹⁹F-NMR measurements of the obtained compound (P) were carried out, and the structure was identified from the following results.

¹H-NMR (CD₃COCD₃): δ [ppm]=1.37 to 1.61 (12H), 3.40 to 4.43 (46H)

¹⁹F-NMR (acetone-d₆): δ [ppm]=−55.6 to −50.6 (10F), −77.7 (4F), −80.3 (4F), −84.0 to −83.0 (10F), −86.4 (4F), −91.0 to −88.5 (20F), −124.3 (4F), −130.0 to −129.0 (5F)

### Example 17

The same operation as in Example 9 was carried out except that a compound represented by HOCH₂CF₂O(CF₂CF₂O)ₛ(CF₂O)ₜCF₂CH₂OH (s and t indicating the average degree of polymerization in the formula are 2.5) (number average molecular weight: 633, molecular weight distribution: 1.1) was used instead of the compound represented by HOCH₂CF₂CF₂O(CF₂CF₂CF₂O)_{q}CF₂CF₂CH₂OH (q indicating the average degree of polymerization in the formula is 2.5) and 3.64 g of the compound represented by Formula (16) above was used instead of the compound represented by Formula (20) in the second reaction, thereby obtaining 4.3 g (molecular weight: 2364, 1.8 mmol) of the compound represented by Formula (Q) above (Fdq₁ and Ffq₁ in Formula (Q) are represented by Formula (QF), qq indicating the average degree of polymerization in Fdq₁ is 2.5, and sq and tq indicating the average degree of polymerization in Ffq₁ are 2.5).

¹H-NMR and ¹⁹F-NMR measurements of the obtained compound (Q) were carried out, and the structure was identified from the following results.

¹H-NMR (CD₃COCD₃): δ [ppm]=1.36 to 1.57 (8H), 3.39 to 4.37 (46H)

¹⁹F-NMR (acetone-d₆): δ [ppm]=−55.6 to −50.6 (10F), −77.7 (4F), −80.3 (4F), −84.0 to −83.0 (10F), −86.4 (4F), −91.0 to −88.5 (20F), −124.3 (4F), −130.0 to −129.0 (5F)

### Example 18

The same operation as in Example 3 was carried out except that 14.0 g of the compound represented by HOCH₂CF₂O(CF₂CF₂O)ᵣCF₂CH₂OH (r indicating the average degree of polymerization in the formula is 2.5) (number average molecular weight: 468, molecular weight distribution: 1.1) was used instead of the compound represented by HOCH₂CF₂O(CF₂CF₂O)ₛ(CF₂O)ₜCF₂CH₂OH (s and t indicating the average degree of polymerization in the formula are 2.5) and 5.77 g of the compound represented by Formula (21) above was used instead of the compound represented by Formula (14) above in the second reaction, thereby obtaining 3.8 g (molecular weight: 2094, 1.8 mmol) of the compound represented by Formula (R) above (Ffr₁ and Fpr₁ in Formula (R) are represented by Formula (RF), sr and tr indicating the average degree of polymerization in

Ffr₁ are 2.5, and rr indicating the average degree of polymerization in Fpr₁ is 2.5).

¹H-NMR and ¹⁹F-NMR measurements of the obtained compound (R) were carried out, and the structure was identified from the following results.

¹H-NMR (CD₃COCD₃): δ [ppm]=1.34 to 1.59 (4H), 3.40 to 4.43 (58H)

¹⁹F-NMR (CD₃COCD₃): δ [ppm]=−55.6 to −50.6 (5F), −77.7 (2F), −78.6 (4F), −80.3 (2F), −81.3 (4F), −90.0 to −88.5 (30F)

### Example 19

The same operation as in Example 3 was carried out except that 20.8 g of the compound represented by HOCH₂CF₂CF₂O(CF₂CF₂CF₂O)_{q}CF₂CF₂CH₂OH (q indicating the average degree of polymerization in the formula is 2.5) (number average molecular weight: 693, molecular weight distribution: 1.1) was used instead of the compound represented by HOCH₂CF₂O(CF₂CF₂O)ₛ(CF₂O)ₜCF₂CH₂OH (s and t indicating the average degree of polymerization in the formula are 2.5) and 3.61 g of the compound represented by Formula (24) below was used instead of the compound represented by Formula (14) above in the second reaction, thereby obtaining 4.3 g (molecular weight: 2392, 1.8 mmol) of the compound represented by Formula (S) above (Fds₁ and Ffs₁ in Formula (S) are represented by Formula (SF), qs indicating the average degree of polymerization in Fds₁ is 2.5, and ss and ts indicating the average degree of polymerization in Ffs₁ are 2.5).

The compound represented by Formula (24) was synthesized by introducing a THP group (tetrahydropyranyl group) into a primary hydroxyl group in 5-hexen-1-ol and oxidizing the double bond of the obtained compound.

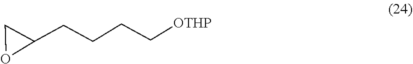

(In Formula (24), THP represents a tetrahydropyranyl group.)

¹H-NMR and ¹⁹F-NMR measurements of the obtained compound (S) were carried out, and the structure was identified from the following results.

¹H-NMR (CD₃COCD₃): δ [ppm]=1.34 to 1.57 (16H), 3.41 to 4.37 (38H)

¹⁹F-NMR (CD₃COCD₃): δ [ppm]=−55.6 to −50.6 (5F), −77.7 (2F), −80.3 (2F), −84.0 to −83.0 (20F), −86.4 (8F), −91.0 to −88.5 (10F), −124.3 (8F), −130.0 to −129.0 (10F)

### Example 20

The same operation as in Example 10 was carried out except that the compound represented by HOCH₂CF₂O(CF₂CF₂O)ᵣCF₂CH₂OH (r indicating the average degree of polymerization in the formula is 2.5) (number average molecular weight: 468, molecular weight distribution: 1.1) was used instead of the compound represented by HOCH₂CF₂O(CF₂CF₂O)ₛ(CF₂O)ₜCF₂CH₂OH (s and t indicating the average degree of polymerization in the formula are 2.5) and 5.48 g of a compound represented by Formula (25) below was used instead of the compound represented by Formula (21) in the second reaction, thereby obtaining 3.76 g (molecular weight: 2090, 1.8 mmol) of the compound represented by Formula (T) above (Fft₁ and Fpt₁ in Formula (T) are represented by Formula (TF), st and tt indicating the average degree of polymerization in Fft₁ are 2.5, and rt indicating the average degree of polymerization in Fpt₁ is 2.5).

The compound represented by Formula (25) was synthesized by the following method.

A secondary hydroxyl group of a compound obtained by reacting the compound represented by Formula (19) with allyl alcohol was protected with a MOM group. The double bond of the obtained compound was oxidized to obtain the compound represented by Formula (25).

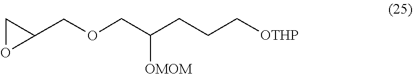

(In Formula (25), THP represents a tetrahydropyranyl group, and MOM represents a methoxymethyl group.)

¹H-NMR and ¹⁹F-NMR measurements of the obtained compound (T) were carried out, and the structure was identified from the following results.

¹H-NMR (CD₃COCD₃): δ [ppm]=1.35 to 1.60 (16H), 3.42 to 4.42 (50H)

¹⁹F-NMR (CD₃COCD₃): δ [ppm]=−55.6 to −50.6 (5F), −77.7 (2F), −78.6 (4F), −80.3 (2F), −81.3 (4F), −90.0 to −88.5 (30F)

### Comparative Example 1

A compound represented by Formula (AA) below was synthesized by the method described in Patent Document 1.

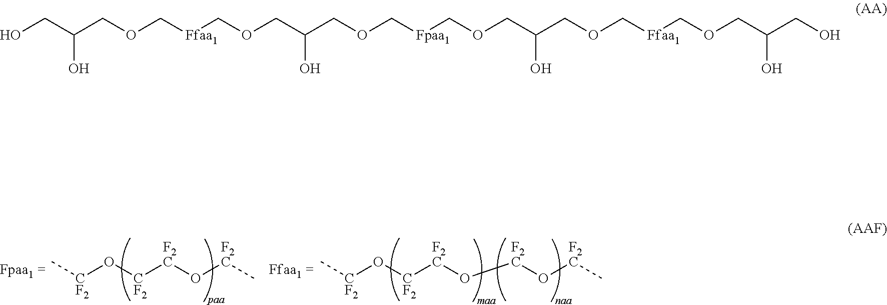

(Fpaa₁ and Ffaa₁ in Formula (AA) are represented by Formula (AAF), paa indicating the average degree of polymerization in Fpaa₁ is 2.5, and maa and naa indicating the average degree of polymerization in Ffaa₁ each independently represent 2.5.)

¹H-NMR and ¹⁹F-NMR measurements of the obtained compound (AA) were carried out, and the structure was identified from the following results.

¹H-NMR (CD₃COCD₃): δ [ppm]=3.42 to 4.28 (38H)

¹⁹F-NMR (CD₃COCD₃): δ [ppm]=−55.6 to −50.6 (10F), −77.7 (4F), −78.6 (2F), −80.3 (4F), −81.3 (2F), −90.0 to −88.5 (30F)

### Comparative Example 2

A compound represented by Formula (AB) below was synthesized by the method described in Patent Document 1.

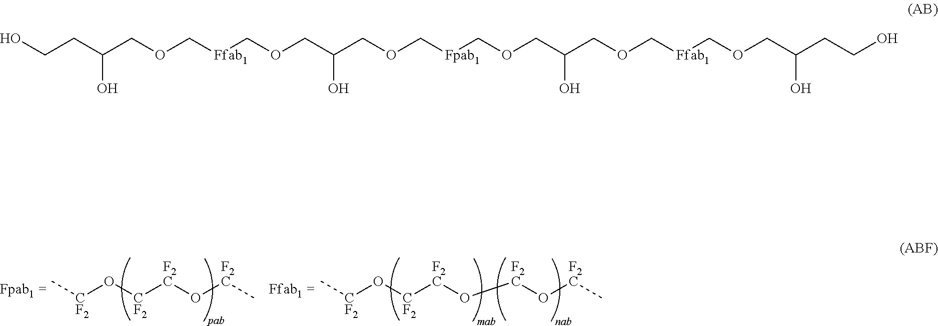

(Fpab₁ and Ffab₁ in Formula (AB) are represented by Formula (ABF), pab indicating the average degree of polymerization in Fpab₁ is 2.5, and mab and nab indicating the average degree of polymerization in Ffab₁ each independently represent 2.5.)

¹H-NMR and ¹⁹F-NMR measurements of the obtained compound (AB) were carried out, and the structure was identified from the following results.

### (Identification Data)

¹H-NMR (CD₃COCD₃): δ [ppm]=1.54 to 1.76 (4H), 3.42 to 4.28 (38H)

¹⁹F-NMR (CD₃COCD₃): δ [ppm]=−55.6 to −50.6 (10F), −77.7 (4F), −78.6 (2F), −80.3 (4F), −81.3 (2F), −90.0 to −88.5 (30F)

### Comparative Example 3

A compound represented by Formula (AC) below was synthesized by the method described in Patent Document 1.

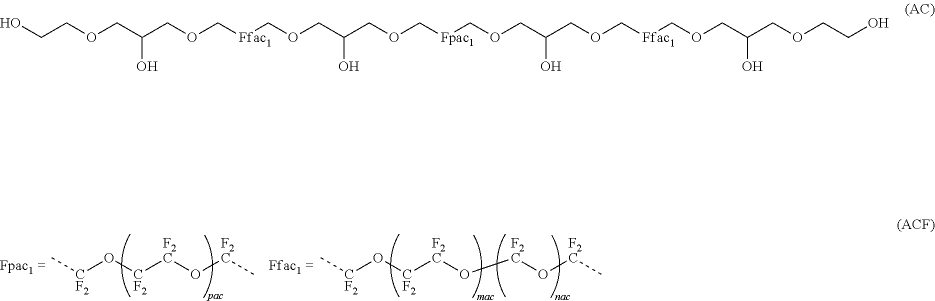

(Fpac₁ and Ffac₁ in Formula (AC) are represented by Formula (ACF), pac indicating the average degree of polymerization in Fpac₁ is 2.5, and mac and nac indicating the average degree of polymerization in Ffac₁ each independently represent 2.5.)

¹H-NMR and ¹⁹F-NMR measurements of the obtained compound (AC) were carried out, and the structure was identified from the following results.

(Identification data) ¹H-NMR (CD₃COCD₃): δ [ppm]=3.46 to 4.18 (46H)

¹⁹F-NMR (CD₃COCD₃): δ [ppm]=−55.6 to −50.6 (10F), −77.7 (4F), −78.6 (2F), −80.3 (4F), −81.3 (2F), −90.0 to −88.5 (30F)

### Comparative Example 4

A compound represented by Formula (AD) below was synthesized by the method described in Patent Document 1.

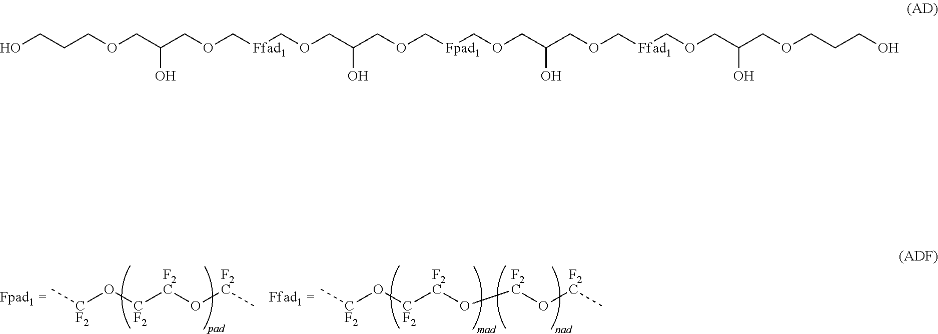

(Fpad₁ and Ffad₁ in Formula (AD) are represented by Formula (ADF), pad indicating the average degree of polymerization in Fpad₁ is 2.5, and mad and nad indicating the average degree of polymerization in Ffad₁ each independently represent 2.5.)

¹H-NMR and ¹⁹F-NMR measurements of the obtained compound (AD) were carried out, and the structure was identified from the following results.

### (Identification Data)

¹H-NMR (CD₃COCD₃): δ [ppm]=1.34 to 1.67 (4H), 3.39 to 4.34 (46H)

¹⁹F-NMR (CD₃COCD₃): δ [ppm]=−55.6 to −50.6 (10F), −77.7 (4F), −78.6 (2F), −80.3 (4F), −81.3 (2F), −90.0 to −88.5 (30F)

### Comparative Example 5

A compound represented by Formula (AE) below was synthesized by the method described in Patent Document 2.

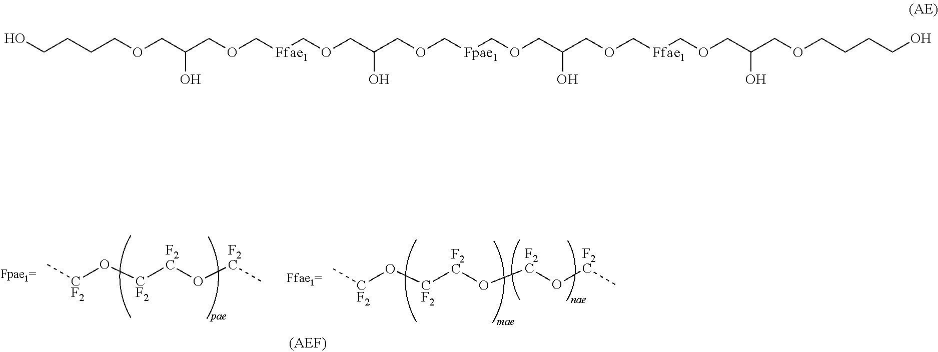

(Fpae₁ and Ffae₁ in Formula (AF) are represented by Formula (AFF), pae indicating the average degree of polymerization in Fpae₁ is 2.5, and mae and nae indicating the average degree of polymerization in Ffae₁ each independently represent 2.5.)

¹H-NMR and ¹⁹F-NMR measurements of the obtained compound (AE) were carried out, and the structure was identified from the following results.

### (Identification Data)

¹H-NMR (CD₃COCD₃): δ [ppm]=1.34 to 1.65 (8H), 3.42 to 4.35 (46H)

¹⁹F-NMR (CD₃COCD₃): δ [ppm]=−55.6 to −50.6 (10F), −77.7 (4F), −78.6 (2F), −80.3 (4F), −81.3 (2F), −90.0 to −88.5 (30F)

### Comparative Example 6

The compound represented by Formula (AF) below was synthesized by the following method.

The same operation as in Example 12 was carried out except that epibromohydrin was used instead of 2-(2-bromoethyl)oxirane in the first reaction and 4.47 g of a compound represented by Formula (26) below was used instead of the compound represented by Formula (22) in the second reaction, thereby obtaining 3.9 g (molecular weight: 2143, 1.8 mmol) of a compound represented by Formula (AF) below.

The compound represented by Formula (26) was obtained by introducing a THP group as a protective group into the primary and secondary hydroxyl groups of 3-allyloxy-1,2-propanediol and oxidizing the double bond of the obtained compound.

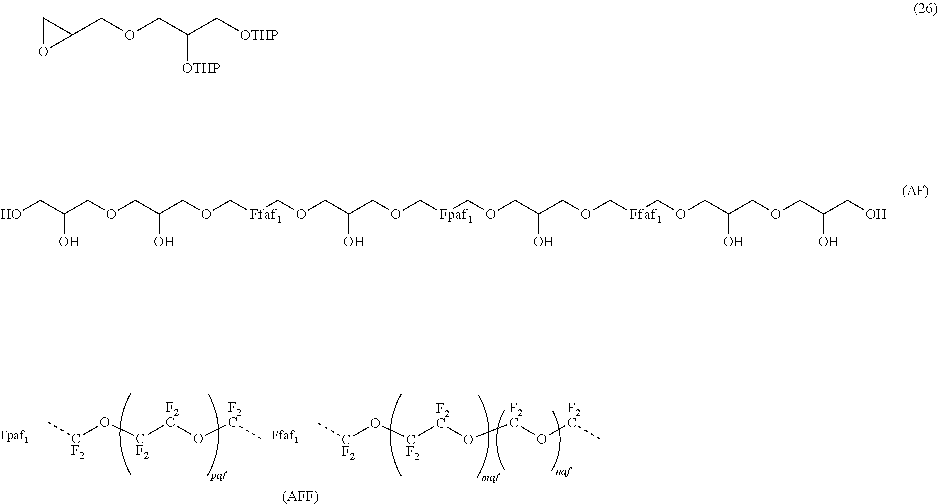

(In Formula (26), THP represents a tetrahydropyranyl group.)

(Fpaf₁ and Ffaf₁ in Formula (AF) are represented by Formula (AFF), paf indicating the average degree of polymerization in Fpaf₁ is 2.5, and maf and naf indicating the average degree of polymerization in Ffaf₁ each independently represent 2.5.)

¹H-NMR and ¹⁹F-NMR measurements of the obtained compound (AF) were carried out, and the structure was identified from the following results.

¹H-NMR (CD₃COCD₃): δ [ppm]=3.42 to 4.35 (50H)

¹⁹F-NMR (CD₃COCD₃): δ [ppm]=−55.6 to −50.6 (10F), −77.7 (4F), −78.6 (2F), −80.3 (4F), −81.3 (2F), −90.0 to −88.5 (30F)

### Comparative Example 7

A compound represented by Formula (AG) below was synthesized by the method described in Patent Document 2.

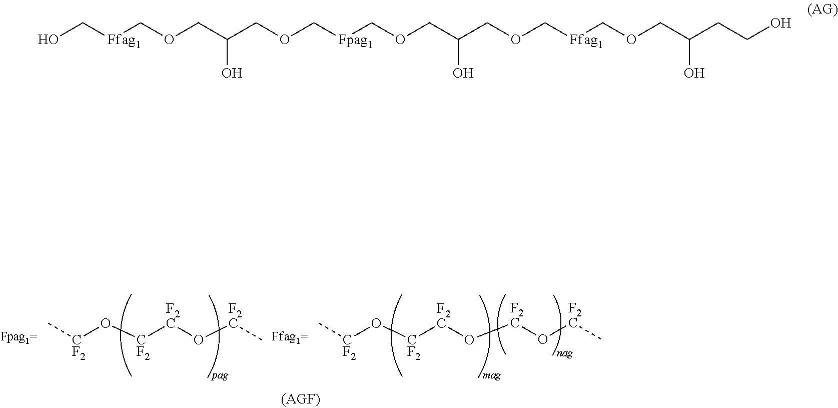

(Fpag₁ and Ffag₁ in Formula (AG) are represented by Formula (AGF), pag indicating the average degree of polymerization in Fpag₁ is 2.5, and mag and nag indicating the average degree of polymerization in Ffag₁ each independently represent 2.5.)

¹H-NMR and ¹⁹F-NMR measurements of the obtained compound (AG) were carried out, and the structure was identified from the following results.

(Identification Data) ¹H-NMR (CD₃COCD₃): δ [ppm]=1.54 to 1.76 (2H), 3.42 to 4.28 (32H)

¹⁹F-NMR (CD₃COCD₃): δ [ppm]=−55.6 to −50.6 (10F), −77.7 (4F), −78.6 (2F), −80.3 (4F), −81.3 (2F), −90.0 to −88.5 (30F)

### Comparative Example 8

A compound represented by Formula (AH) below was synthesized by the method described in Patent Document 1.

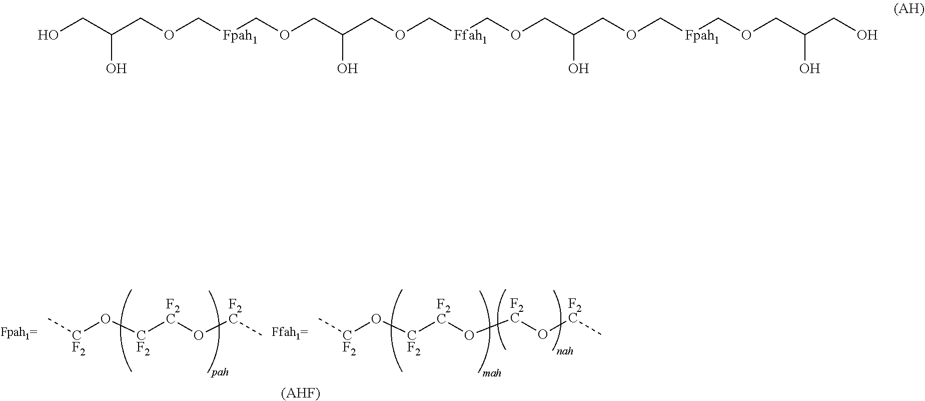

(Fpah₁ and Ffah₁ in Formula (AH) are represented by Formula (AHF), pah indicating the average degree of polymerization in Fpah₁ is 2.5, and mah and nah indicating the average degree of polymerization in Ffah₁ each independently represent 2.5.)

¹H-NMR and ¹⁹F-NMR measurements of the obtained compound (AH) were carried out, and the structure was identified from the following results.

(Identification data)

¹H-NMR (CD₃COCD₃): δ [ppm]=3.42 to 4.28 (38H)

¹⁹F-NMR (CD₃COCD₃): δ [ppm]=−55.6 to −50.6 (5F), −77.7 (2F), −78.6 (4F), −80.3 (2F), −81.3 (4F), −90.0 to −88.5 (30F)

### Comparative Example 9

A compound represented by Formula (AI) below was synthesized by the method described in Patent Document 1.

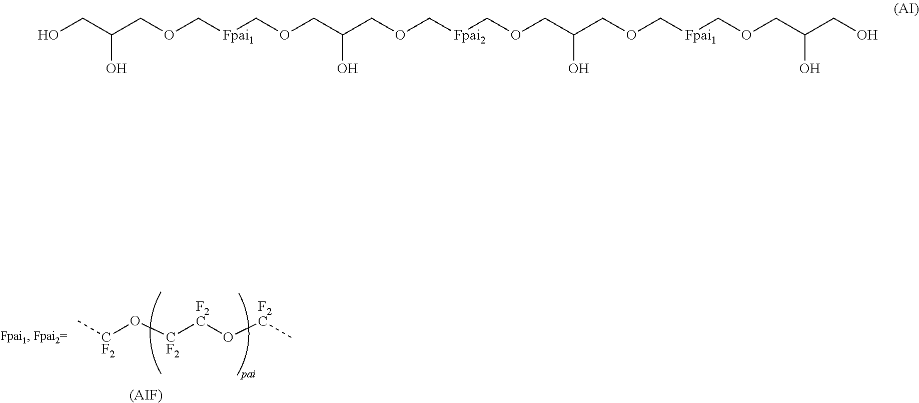

(Fpa₁ and Fpa₂ in Formula (AI) are represented by Formula (AIF), pai indicating the average degree of polymerization in Fpai₁ is 2.5, and pai indicating the average degree of polymerization in Fpa₂ is 2.5.)

¹H-NMR and ¹⁹F-NMR measurements of the obtained compound (AI) were carried out, and the structure was identified from the following results.

### (Identification Data)

¹H-NMR (CD₃COCD₃): δ [ppm]=3.42 to 4.28 (38H)

¹⁹F-NMR (CD₃COCD₃): δ [ppm]=−78.6 (6F), −81.3 (6F), −90.0 to −88.5 (30F)

### Comparative Example 10

A compound represented by Formula (AJ) below was synthesized by the method described in Patent Document 1.

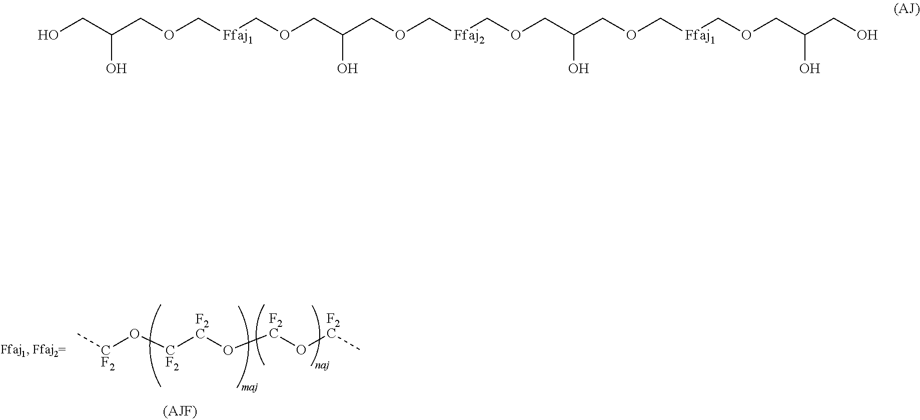

(Ffaj₁ and Ffaj₂ in Formula (AJ) are represented by Formula (AJF), maj and naj indicating the average degree of polymerization in Ffaj₁ is 2.5. and maj and naj indicating the average degree of polymerization in Ffaj₂ represent 2.5.)

¹H-NMR and ¹⁹F-NMR measurements of the obtained compound (AJ) were carried out, and the structure was identified from the following results.

### (Identification Data)

¹H-NMR (CD₃COCD₃): δ [ppm]=3.42 to 4.28 (38H)

¹⁹F-NMR (CD₃COCD₃): δ [ppm]=−55.6 to −50.6 (15F), −77.7 (6F), −80.3 (6F), −91.0 to −88.5 (30F)

### Comparative Example 11

A compound represented by Formula (AK) below was synthesized by the method described in Patent Document 2.

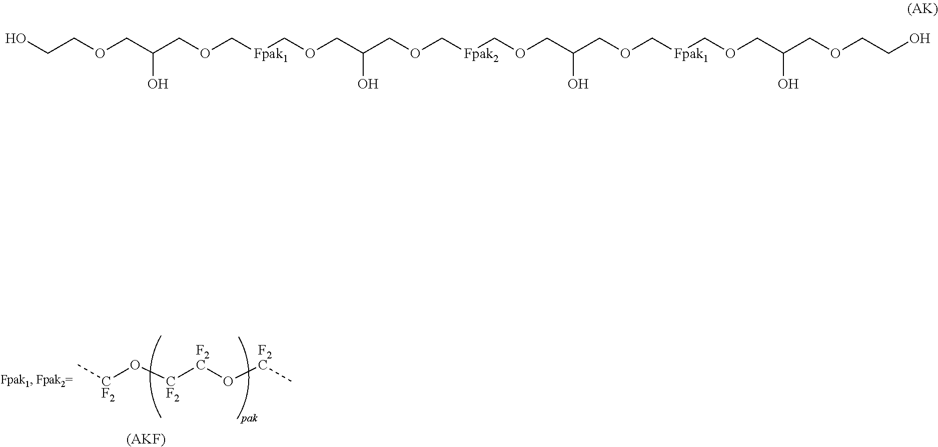

(Fpak₁ and Fpak₂ in Formula (AK) are represented by Formula (AKF), pak indicating the average degree of polymerization in Fpak₁ is 2.5, and pak indicating the average degree of polymerization in Fpak₂ represents 2.5.)

¹H-NMR and ¹⁹F-NMR measurements of the obtained compound (AK) were carried out, and the structure was identified from the following results.

### (Identification Data)

¹H-NMR (CD₃COCD₃): δ [ppm]=¹H-NMR (CD₃COCD₃): δ [ppm]=3.46 to 4.18 (46H)

¹⁹F-NMR (CD₃COCD₃): δ [ppm]=−78.6 (6F), −81.3 (6F), −90.0 to −88.5 (30F)

### Comparative Example 12

A compound represented by Formula (AL) below was synthesized by the method described in Patent Document 2.

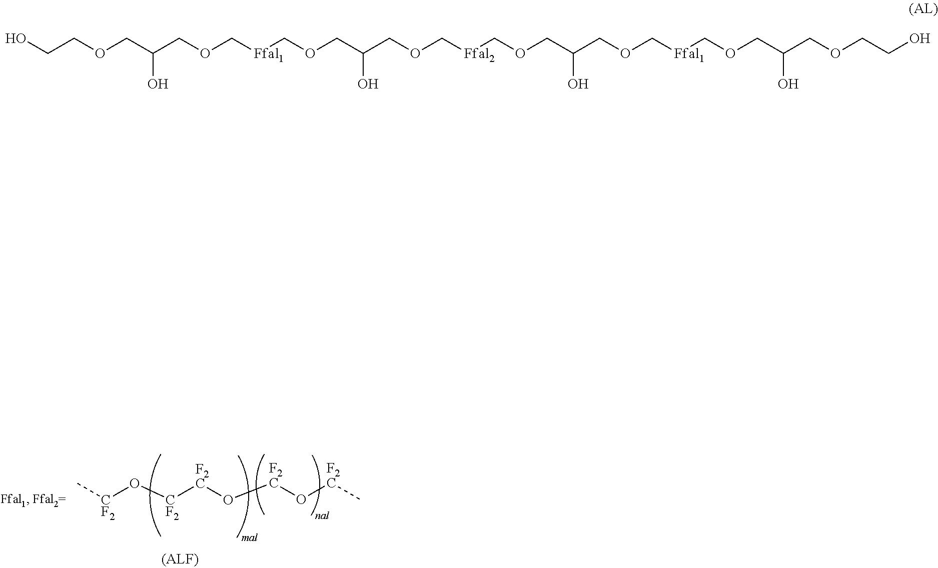

(Ffal₁ and Ffal₂ in Formula (AL) are represented by Formula (ALF), mal and nal indicating the average degree of polymerization in Ffal₁ are 2.5, and mal and nal indicating the average degree of polymerization in Ffal₂ represent 2.5.)

¹H-NMR and ¹⁹F-NMR measurements of the obtained compound (AL) were carried out, and the structure was identified from the following results.

### (Identification Data)

¹H-NMR (CD₃COCD₃): δ [ppm]=¹H-NMR (CD₃COCD₃): δ [ppm]=3.46 to 4.18 (46H)

¹⁹F-NMR (CD₃COCD₃): δ [ppm]=−55.6 to −50.6 (15F), −77.7 (6F), −80.3 (6F), −91.0 to −88.5 (30F)

### Comparative Example 13

A compound represented by Formula (AM) below was synthesized by the method described in Patent Document 2.

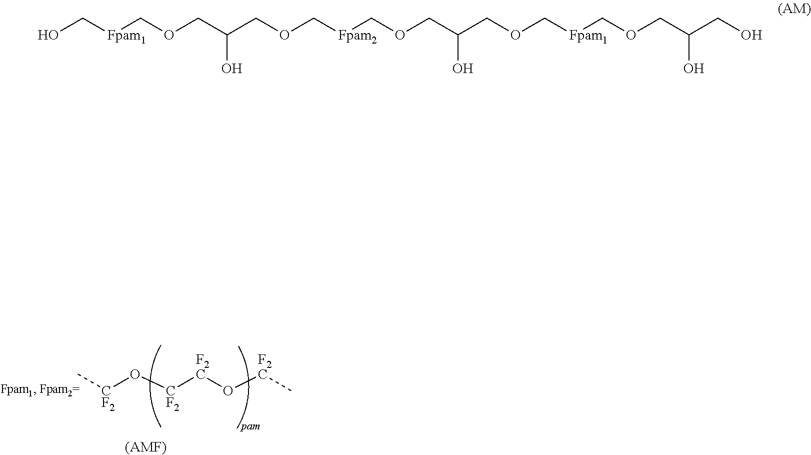

(Fpam₁ and Fpam₂ in Formula (AM) are represented by Formula (AMF), pam indicating the average degree of polymerization in Fpam₁ is 2.5, and pam indicating the average degree of polymerization in Fpam₂ represents 2.5.)

¹H-NMR and ¹⁹F-NMR measurements of the obtained compound (AM) were carried out, and the structure was identified from the following results.

(Identification data) ¹H-NMR (CD₃COCD₃): δ [ppm]=3.46 to 4.18 (32H)

¹⁹F-NMR (CD₃COCD₃): δ [ppm]=−78.6 (6F), −81.3 (6F), −90.0 to −88.5 (30F)

### Comparative Example 14

The compound represented by Formula (AN) below was synthesized by the following method.

14.0 g of the compound represented by HOCH₂CF₂O(CF₂CF₂O)ᵣCF₂CH₂OH (r indicating the average degree of polymerization in the formula is 2.5) (number average molecular weight: 468, molecular weight distribution: 1.1), 2.34 g (molecular weight: 130.19, 18 mmol) of tert-butyl glycidyl ether, and 28 mL of t-butanol were added to a 200 mL eggplant flask in a nitrogen gas atmosphere and stirred until the mixture became uniform at room temperature. 1.0 g (molecular weight: 112.2, 9 mmol) of potassium tert-butoxide was further added to this uniform solution and reacted by being stirred at 70° C. for 16 hours.

A reaction product obtained after the reaction was cooled to 25° C., moved to a separatory funnel containing 100 mL of water, and extracted three times with 100 mL of ethyl acetate. An organic layer thereof was washed with water and dehydrated with anhydrous sodium sulfate. After the drying agent was filtered, the filtrate was concentrated, and the residue was purified by silica gel column chromatography, thereby obtaining 7.2 g of a compound represented by Formula (27) below (molecular weight: 598, 12.0 mmol).

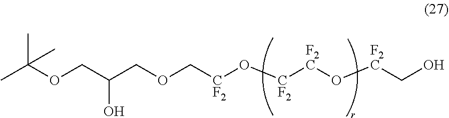

(In Formula (27), r indicating the average degree of polymerization is 2.5.)

7.2 g of the compound represented by Formula (27) (r indicating the average degree of polymerization in the formula is 2.5), 0.67 g of potassium tert-butoxide, and 10.5 mL of t-butanol were added to a 200 mL eggplant flask in a nitrogen gas atmosphere and stirred until the mixture became uniform at room temperature. 2.60 g of a compound represented by Formula (28) below was further added to this uniform liquid and reacted by being stirred at 70° C. for 16 hours.

The compound represented by Formula (28) was synthesized by oxidizing diallyl ether.

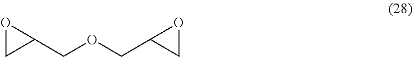

A reaction product obtained after the reaction was cooled to 25° C., moved to a separatory funnel containing 100 ml of water, and extracted three times with 100 mL of ethyl acetate. An organic layer thereof was washed with water and dehydrated with anhydrous sodium sulfate. After the drying agent was filtered, the filtrate was concentrated, and the residue was purified by silica gel column chromatography, thereby obtaining 5.9 g of a compound represented by Formula (29) below (molecular weight: 728, 7.7 mmol).

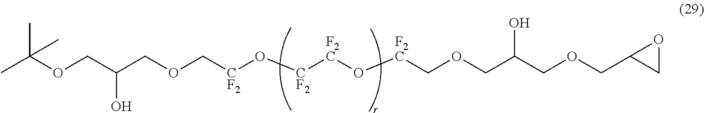

(In Formula (29), r indicating the average degree of polymerization is 2.5.)

5.9 g of the compound represented by Formula (29) (r indicating the average degree of polymerization in the formula is 2.5), 0.12 g of potassium tert-butoxide, and 2.8 mL of t-butanol were added to a 200 mL eggplant flask in a nitrogen gas atmosphere and stirred until the mixture became uniform at room temperature. 1.6 g of the compound represented by HOCH₂CF₂O(CF₂CF₂O)ᵣCF₂CH₂OH (r indicating the average degree of polymerization in the formula is 2.5) (number average molecular weight: 468, molecular weight distribution: 1.1) was further added to this uniform liquid and reacted by being stirred at 70° C. for 16 hours.

A reaction product obtained after the reaction was cooled to 25° C., moved to a separatory funnel containing 100 mL of water, and extracted three times with 100 mL of ethyl acetate. An organic layer thereof was washed with water and dehydrated with anhydrous sodium sulfate. After the drying agent was filtered, the filtrate was concentrated, and the residue was purified by silica gel column chromatography, thereby obtaining 3.3 g of a compound represented by Formula (AN) below (molecular weight: 1812, 2.4 mmol).

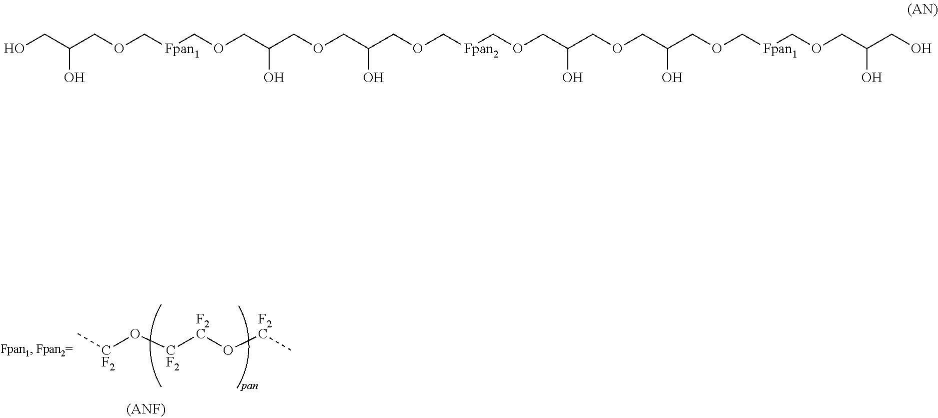

(Fpan₁ and Fpan₂ in Formula (AN) are represented by Formula (ANF), pan indicating the average degree of polymerization in Fpan₁ is 2.5, and pan indicating the average degree of polymerization in Fpan₂ represents 2.5.)

¹H-NMR and ¹⁹F-NMR measurements of the obtained compound (AN) were carried out, and the structure was identified from the following results.

¹H-NMR (CD₃COCD₃): δ [ppm]=3.47 to 4.24 (50H)

¹⁹F-NMR (CD₃COCD₃): δ [ppm]=−78.6 (6F), −81.3 (6F), −90.0 to −88.5 (30F)

Structures of R¹ and R⁷ (e and f in Formula (4-1), g and h in Formula (4-2), i in Formula (4-3), j in Formula (4-4), and Formula (4-5)), structures of R³ and R⁵ (a and b in Formula (2) and c and d in Formula (3)), and structures of R², R⁴, and R⁶ (k and l in Formula (5), m in Formula (6), and n in Formula (7)) when the thus obtained compounds of Examples 1 to 20 and Comparative Examples 1 to 14 are applied to Formula (1) are shown in Tables 1 and 2.

TABLE 1

| | R¹, R⁷ | R², R⁶ | R⁴ | R³ | R⁵ | Compound |
|---|---|---|---|---|---|---|
| Example 1 | Formula (4-2) | Formula (6) | Formula (6) | Formula (2) | Formula (3) | (A) |
| | g = 1, h = 2 | m = 2.5 | m = 2.5 | a = 2, b = 1 | c = 2, d = 1 | |
| Example 2 | Formula (4-2) | Formula (7) | Formula (7) | Formula (2) | Formula (3) | (B) |
| | g = 2, h = 2 | n = 2.5 | n = 2.5 | a = 2, b = 1 | c = 2, d = 1 | |
| Example 3 | Formula (4-1) | Formula (5) | Formula (5) | Formula (2) | Formula (3) | (C) |
| | e = 0, f = 1 | k = 2.5 | k = 2.5 | a = 2, b = 1 | c = 2, d = 1 | |
| | | l = 2.5 | l = 2.5 | | | |
| Example 4 | Formula (4-5) | Formula (6) | Formula (6) | Formula (2) | Formula (3) | (D) |
| | | m = 2.5 | m = 2.5 | a = 2, b = 1 | c = 2, d = 1 | |
| Example 5 | Formula (4-2) | Formula (7) | Formula (7) | Formula (2) | Formula (3) | (E) |
| | g = 1, h = 1 | n = 2.5 | n = 2.5 | a = 2, b = 1 | c = 2, d = 1 | |
| Example 6 | Formula (4-3) | Formula (7) | Formula (7) | Formula (2) | Formula (3) | (F) |
| | i = 1 | n = 2.5 | n = 2.5 | a = 2, b = 1 | c = 2, d = 1 | |
| Example 7 | Formula (4-1) | Formula (5) | Formula (5) | Formula (2) | Formula (3) | (G) |
| | c = 1, f = 1 | k = 2.5 | k = 2.5 | a = 2, b = 1 | c = 2, d = 1 | |
| | | l = 2.5 | l = 2.5 | | | |
| Example 8 | Formula (4-1) | Formula (6) | Formula (6) | Formula (2) | Formula (3) | (H) |
| | e = 0, f = 2 | m = 2.5 | m = 2.5 | a = 3, b = 1 | c = 3, d = 1 | |
| Example 9 | Formula (4-4) | Formula (7) | Formula (7) | Formula (2) | Formula (3) | (I) |
| | j = 2 | n = 2.5 | n = 2.5 | a = 3, b = 1 | c = 3, d = 1 | |
| Example 10 | Formula (4-2) | Formula (5) | Formula (5) | Formula (2) | Formula (3) | (J) |
| | g = 2, h = 1 | k = 2.5 | k = 2.5 | a = 3, b = 1 | c = 3, d = 1 | |
| | | l = 2.5 | l = 2.5 | | | |
| Example 11 | Formula (4-1) | Formula (7) | Formula (6) | Formula (2) | Formula (3) | (K) |
| | e = 1, f = 1 | n = 2.5 | m = 2.5 | a = 2, b = 1 | c = 2, d = 1 | |
| Example 12 | Formula (4-4) | Formula (5) | Formula (6) | Formula (2) | Formula (3) | (L) |
| | j = 1 | k = 2.5 | m = 2.5 | a = 2, b = 1 | c = 2, d = 1 | |
| | | l = 2.5 | | | | |
| Example 13 | Formula (4-2) | Formula (7) | Formula (6) | Formula (2) | Formula (3) | (M) |
| | g = 1, h = 2 | n = 2.5 | m = 2.5 | a = 3, b = 1 | c = 3, d = 1 | |
| Example 14 | Formula (4-2) | Formula (6) | Formula (7) | Formula (2) | Formula (3) | (N) |
| | g = 2, h = 2 | m = 2.5 | n = 2.5 | a = 2, b = 1 | c = 2, d = 1 | |
| Example 15 | Formula (4-5) | Formula (6) | Formula (7) | Formula (2) | Formula (3) | (O) |
| | | m = 2.5 | n = 2.5 | a = 2, b = 1 | c = 2, d = 1 | |
| Example 16 | Formula (4-2) | Formula (5) | Formula (7) | Formula (2) | Formula (3) | (P) |
| | g = 1, h = 3 | k = 2.5 | n = 2.5 | a = 2, b = 1 | c = 2, d = 1 | |
| | | l = 2.5 | | | | |
| Example 17 | Formula (4-2) | Formula (5) | Formula (7) | Formula (2) | Formula (3) | (Q) |
| | g = 1, h = 1 | k = 2.5 | n = 2.5 | a = 3, b = 1 | c = 3, d = 1 | |
| | | l = 2.5 | | | | |
| Example 18 | Formula (4-2) | Formula (6) | Formula (5) | Formula (2) | Formula (3) | (R) |
| | g = 2, h = 1 | m = 2.5 | k = 2.5 | a = 2, b = 1 | c = 2, d = 1 | |
| | | | l = 2.5 | | | |
| Example 19 | Formula (4-1) | Formula (7) | Formula (5) | Formula (2) | Formula (3) | (S) |
| | e = 0, f = 3 | n = 2.5 | k = 2.5 | a = 2, b = 1 | c = 2, d = 1 | |
| | | | l = 2.5 | | | |
| Example 20 | Formula (4-1) | Formula (6) | Formula (5) | Formula (2) | Formula (3) | (T) |
| | e = 1, f = 2 | m = 2.5 | k = 2.5 | a = 3, b = 1 | c = 3, d = 1 | |
| | | | l = 2.5 | | | |

TABLE 2

| | —R¹ | R², R⁶ | R⁴ | R³ | R⁵ | —R⁷ | Compound |
|---|---|---|---|---|---|---|---|
| Comparative Example 1 | 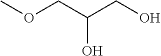 | Formula (5) k = 2.5 l = 2.5 | Formula (6) m = 2.5 | Formula (2) a = 1, b = 1 | Formula (3) c = 1, d = 1 | same as —R¹ | (AA) |
| Comparative | Formula (4-1) | Formula | Formula | Formula | Formula | same as —R¹ | (AB) |
| Example 2 | c = 0, f = 1 | (5) | (6) | (2) | (3) | | |
| | | k = 2.5 | m = 2.5 | a = 1, | c = 1, | | |
| | | l = 2.5 | | b = 1 | d = 1 | | |
| Comparative | Formula (4-2) | Formula | Formula | Formula | Formula | same as —R¹ | (AC) |
| Example 3 | g = 1, h = 1 | (5) | (6) | (2) | (3) | | |
| | | k = 2.5 | m = 2.5 | a = 1, | c = 1, | | |
| | | l = 2.5 | | b = 1 | d = 1 | | |
| Comparative | Formula (4-2) | Formula | Formula | Formula | Formula | same as —R¹ | (AD) |
| Example 4 | g = 1, h = 2 | (5) | (6) | (2) | (3) | | |
| | | k = 2.5 | m = 2.5 | a = 1, | c = 1, | | |
| | | l = 2.5 | | b = 1 | d = 1 | | |
| Comparative | Formula (4-2) | Formula | Formula | Formula | Formula | same as —R¹ | (AE) |
| Example 5 | g = 1, h = 3 | (5) | (6) | (2) | (3) | | |
| | | k = 2.5 | m = 2.5 | a = 1, | c = 1, | | |
| | | l = 2.5 | | b = 1 | d = 1 | | |
| Comparative Example 6 | 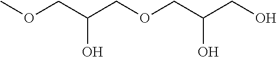 | Formula (5) k = 2.5 l = 2.5 | Formula (6) m = 2.5 | Formula (2) a = 1, b = 1 | Formula (3) c = 1, d = 1 | same as —R¹ | (AF) |
| Comparative | —OH | Formula | Formula | Formula | Formula | Formula (4-1) | (AG) |
| Example 7 | | (5) | (6) | (2) | (3) | e = 0, f = 1 | |
| | | k = 2.5 | m = 2.5 | a = 1, | c = 1, | | |
| | | l = 2.5 | | b = 1 | d = 1 | | |
| Comparative Example 8 | 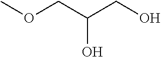 | Formula (6) m = 2.5 | Formula (5) k = 2.5 l = 2.5 | Formula (2) a = 1, b = 1 | Formula (3) c = 1, d = 1 | same as —R¹ | (AH) |
| Comparative Example 9 | 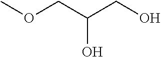 | Formula (6) m = 2.5 | Formula (6) m = 2.5 | Formula (2) a = 1, b = 1 | Formula (3) c = 1, d = 1 | same as —R¹ | (AI) |
| Comparative Example 10 | 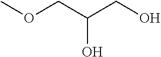 | Formula (5) k = 2.5 l = 2.5 | Formula (5) k = 2.5 l = 2.5 | Formula (2) a = 1, b = 1 | Formula (3) c = 1, d = 1 | same as —R¹ | (AJ) |
| Comparative | Formula (4-2) | Formula | Formula | Formula | Formula | same as —R¹ | (AK) |
| Example 11 | g = 1, h = 1 | (6) | (6) | (2) | (3) | | |
| | | m = 2.5 | m = 2.5 | a = 1, | c = 1, | | |
| | | | | b = 1 | d = 1 | | |
| Comparative | Formula (4-2) | Formula | Formula | Formula | Formula | same as —R¹ | (AL) |
| Example 12 | g = 1, h = 1 | (5) | (5) | (2) | (3) | | |
| | | k = 2.5 | k = 2.5 | a = 1, | c = 1, | | |
| | | l = 2.5 | l = 2.5 | b = 1 | d = 1 | | |
| Comparative Example 13 | —OH | Formula (6) m = 2.5 | Formula (6) m = 2.5 | Formula (2) a = 1, b = 1 | Formula (3) c = 1, d = 1 | 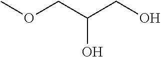 | (AM) |
| Comparative Example 14 | 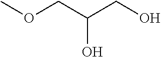 | Formula (6) m = 2.5 | Formula (6) m = 2.5 | Formula (2) a = 1, b = 2 | Formula (3) c = 1, d = 2 | same as —R¹ | (AN) |

In addition, the number average molecular weights (Mn) of the compounds of Examples 1 to 20 and Comparative Examples 1 to 14 were obtained by the above-described ¹H-NMR and ¹⁹F-NMR measurement. The results are shown in Table 3. It is inferred that, in the values of the average molecular weight of the synthesized compounds, variations of approximately 1 to 5 may exist depending on, for example, the molecular weight distributions of the fluoropolyether used as a raw material of the compounds and differences in the operation at the time of synthesizing the compounds.

TABLE 3

| | | Number average | Film | Corrosion resistance test | |
|---|---|---|---|---|---|
| | Compound | molecular weight (Mn) | thickness (Å) | Burnished | Unburnished |
| Example 1 | (A) | 1809 | 10 | B | A |
| Example 2 | (B) | 2632 | 10 | A | A |
| Example 3 | (C) | 2216 | 10 | B | A |
| Example 4 | (D) | 1897 | 10 | A | A |
| Example 5 | (E) | 2456 | 10 | B | A |
| Example 6 | (F) | 2484 | 11 | A | A |
| Example 7 | (G) | 2364 | 10 | A | A |
| Example 8 | (H) | 1777 | 10 | A | A |
| Example 9 | (I) | 2688 | 11 | A | A |
| Example 10 | (J) | 2452 | 10 | A | A |
| Example 11 | (K) | 2319 | 11 | A | A |
| Example 12 | (L) | 2287 | 10 | A | A |
| Example 13 | (M) | 2287 | 10 | A | A |
| Example 14 | (N) | 2182 | 10 | A | A |
| Example 15 | (O) | 2122 | 10 | A | A |
| Example 16 | (P) | 2392 | 10 | A | A |
| Example 17 | (Q) | 2364 | 10 | A | A |
| Example 18 | (R) | 2094 | 10 | A | A |
| Example 19 | (S) | 2392 | 10 | A | A |
| Example 20 | (T) | 2090 | 10 | A | A |
| Comparative Example 1 | (AA) | 1994 | 10 | E | E |
| Comparative Example 2 | (AB) | 2023 | 10 | C | C |
| Comparative Example 3 | (AC) | 2083 | 10 | E | E |
| Comparative Example 4 | (AD) | 2111 | 10 | D | C |
| Comparative Example 5 | (AE) | 2139 | 10 | C | C |
| Comparative Example 6 | (AF) | 2143 | 11 | E | D |
| Comparative Example 7 | (AG) | 1934 | 10 | E | E |
| Comparative Example 8 | (AH) | 1829 | 11 | E | E |
| Comparative Example 9 | (AI) | 1664 | 10 | E | E |
| Comparative Example 10 | (AJ) | 2160 | 10 | E | E |
| Comparative Example 11 | (AK) | 1752 | 10 | D | D |
| Comparative Example 12 | (AL) | 2248 | 11 | D | D |
| Comparative Example 13 | (AM) | 1590 | 10 | E | E |
| Comparative Example 14 | (AN) | 1812 | 11 | E | E |

Next, solutions for forming a lubricating layer were prepared using the compounds obtained in Examples 1 to 20 and Comparative Examples 1 to 14 by a method shown below. Moreover, lubricating layers of magnetic recording media were formed using the obtained solutions for forming a lubricating layer by a method shown below, and magnetic recording media of Examples 1 to 20 and Comparative Examples 1 to 14 were obtained.

### “Solutions for Forming Lubricating Layer”

The compounds obtained in Examples 1 to 20 and Comparative Examples 1 to 14 were each dissolved in VERTREL (registered trademark) XF (trade name, manufactured by Dupont-Mitsui Fluorochemicals Co., Ltd.), which is a fluorine-based solvent, diluted with VERTREL XF such that the film thicknesses of the coating films became 9 Å to 11 Å when applied onto protective layers, and used as solutions for forming a lubricating layer.

### “Magnetic Recording Media”

Magnetic recording media each having an adhesive layer, a soft magnetic layer, a first underlayer, a second underlayer, a magnetic layer, and a protective layer sequentially provided on a substrate having a diameter of 65 mm were prepared. As the protective layer, a carbon layer with a thickness of 1 to 5 nm was used.

The solutions for forming a lubricating layer of Examples 1 to 20 and Comparative Examples 1 to 14 were each applied by the dipping method onto the protective layers of the magnetic recording media in which the individual layers up to the protective layer had been formed. The dipping method was carried out under conditions of an immersion speed of 10 mm/sec, an immersion time of 30 seconds and a lifting speed of 1.2 mm/sec.

Thereafter, a burnishing step was performed in which burnishing tape holding abrasive grains having a grain size #6000 was scanned on the surface of each of the magnetic recording media on which the lubricating layer was formed.

The magnetic recording media after the burnishing step were placed in a thermostatic chamber at 120° C. to perform a heat treatment for 10 minutes.

Magnetic recording media (which were burnished) of Examples 1 to 20 and Comparative Examples 1 to 14 were obtained through the above-described steps.

In addition, magnetic recording media (which were unburnished) of Examples 1 to 20 and Comparative Examples 1 to 14 were obtained in the same manner as the burnished magnetic recording media except that a burnishing step was not performed.

### (Film Thickness Measurement)

The film thicknesses of the lubricating layers in the magnetic recording media (which were burnished and unburnished) of Examples 1 to 20 and Comparative Examples 1 to 14 thus obtained were measured using a Fourier transform infrared spectrophotometer (FT-IR) (trade name: Nicolet iS50, manufactured by Thermo Fisher Scientific Inc.). In all of the magnetic recording media of Examples 1 to 20 and Comparative Examples 1 to 14, there was no difference in the film thickness of the lubricating layer between being burnished and being unburnished. The results are shown in Table 3.

Next, corrosion resistance tests shown below were performed on the burnished and unburnished magnetic recording media of Examples 1 to 20 and Comparative Examples 1 to 14.

### (Corrosion Resistance Tests)

The magnetic recording media were exposed to conditions of 85° C. and a relative humidity of 90% for 48 hours. Thereafter, number of corroded spots of the magnetic recording media was counted using an optical surface analyzer and evaluated based on the following evaluation criteria. The results are shown in Table 3.

### “Evaluation Criteria”

A: Less than 120
B: Greater than or equal to 120 and less than 150
C: Greater than or equal to 150 and less than 350
D: Greater than or equal to 350 and less than 1,000
E: Greater than or equal to 1,000

As shown in Table 3, the magnetic recording media of Examples 1 to 20 having a lubricating layer containing the compound represented by Formula (1) showed a favorable corrosion resistance with the corrosion resistance test results being A in the case where the magnetic recording media were not subjected to tape burnishing and A or B in the case where the magnetic recording media were subjected to tape burnishing.

On the other hand, compared to the magnetic recording media of Examples 1 to 20, the magnetic recording media of Comparative Examples 1 to 14 showed an inferior corrosion resistance with the corrosion resistance test results being C to E in both cases where the magnetic recording media were subjected to and not subjected to tape burnishing.

More specifically, in the magnetic recording media of Examples 1 to 20, lubricating layers contain compounds in which R³ in Formula (1) is Formula (2) (in Formula (2), a is 2 and b is 1) and R⁵ is Formula (3) (in Formula (3), c is 2 and d is 1) or R³ is Formula (2) (in Formula (2), a is 3 and b is 1) and R⁵ is Formula (3) (in Formula (3), c is 3 and d is 1), and R¹ and R⁷ are terminal groups represented by Formulae (4-1) to (4-5). For this reason, the magnetic recording media of Examples 1 to 20 have a larger number of carbon atoms contained in R³ and R⁵ of the compounds in the lubricating layers and more favorable hydrophobicity than the magnetic recording media of Comparative Examples 1 to 14. Moreover, due to the hydrophobicity of the carbon atoms to which the polar groups contained in R¹ and R⁷ are bound and the carbon atoms contained in linking groups in R¹ and R⁷, these lubricating layers have appropriate hydrophobicity. Furthermore, these lubricating layers have favorable adhesion properties with respect to protective layers due to the polar groups in R³, R⁵, R¹, and R⁷. Thus, it is inferred that, in the magnetic recording media of Examples 1 to 20, water was prevented from intruding into the lubricating layers from outside and a favorable corrosion suppressing effect was obtained.

Furthermore, in the magnetic recording media of Examples 8 to 10, 13, 17, and 20, the corrosion resistance test results showed A in all cases of being burnished and unburnished, thereby showing a particularly excellent corrosion suppressing effect. It is inferred that this may be because the lubricating layers of the magnetic recording media of Examples 8 to 10, 13, 17, and 20 contained compounds in which R³ is Formula (2) (in Formula (2), a is 3 and b is 1) and R⁵ is Formula (3) (in Formula (3), c is 3 and d is 1), thereby having a larger number of carbon atoms (longer methylene chain) in R³ and R⁵ of the compounds and more favorable hydrophobicity.

For example, when comparing magnetic recording media with each other which have the same terminal group represented by R¹ and R⁷ and different linking groups represented by R³ and R⁵ (Comparative Example 4 with respect to Examples 1 and 13; Comparative Example 2 with respect to Example 3; Comparative Examples 3, 11, and 12 with respect to Examples 5 and 17; Comparative Example 5 with respect to Example 16), the larger the number of carbon atoms contained in R³ and R⁵ (the longer the methylene chain), the higher the corrosion suppressing effect is exhibited.

In addition, the magnetic recording media of Examples 3, 4, 7, 8, 11, 15, 19, and 20 in which R¹ and R⁷ are terminal groups represented by Formulae (4-1) and (4-5) in which a linking group between a carbon atom to which a terminal polar group is bound and a carbon atom to which a polar group adjacent to the terminal polar group is bound does not contain an ether bond (—O—) showed a favorable corrosion suppressing effect. It is inferred that this may because the linking groups contained in the terminal groups represented by Formulae (4-1) and (4-5) do not contain an ether bond and contain an appropriate number of carbon atoms, and therefore a lubricating layer having favorable hydrophobicity was obtained and the intrusion of water was prevented.

In addition, the magnetic recording media of Examples 1, 2, 5, 6, 9, 10, 12 to 14, and 16 to 18 in which R¹ and R⁷ are terminal groups represented by Formulae (4-2), (4-3), and (4-4) in which a linking group between a carbon atom to which a terminal polar group is bound and a carbon atom to which a polar group adjacent to the terminal polar group is bound contains an ether bond showed a favorable corrosion suppressing effect. It is inferred that this may be because the linking groups contained in the terminal groups represented by Formulae (4-2), (4-3), and (4-4) contain an appropriate number of carbon atoms, and therefore a lubricating layer having favorable hydrophobicity was obtained and the intrusion of water was prevented.

Furthermore, in Examples 2, 4, 7, 9 to 12, 14, 15, 18, and 20 in which R¹ and R⁷ in Formula (1) each have three hydroxyl groups, the corrosion resistance test results showed A in all cases of being burnished and unburnished, thereby showing a particularly excellent corrosion suppressing effect. It is thought that this is because the three hydroxyl groups each contained in R¹ and R⁷ in Formula (1) exhibited excellent adhesion properties with respect to protective layers. If the adhesion properties between a lubricating layer and a protective layer are excellent, water can be prevented from intruding into the lubricating layer from outside.

On the other hand, in Comparative Example 6, although R¹ and R⁷ in Formula (1) each have three hydroxyl groups, the corrosion resistance test results showed E (burnished) and D (unburnished). It is inferred that this may be because, in the compound contained in the lubricating layer in the magnetic recording medium of Comparative Example 6, R³ in Formula (1) is Formula (2) (in Formula (2), a is 1 and b is 1), R⁵ is Formula (3) (in Formula (3), c is 1 and d is 1), and a carbon atom to which a terminal hydroxyl group contained in R¹ and R⁷ is bound is bound to a carbon atom to which a hydroxyl group adjacent to the terminal hydroxyl group is bound. In the lubricating layer containing such a compound, either the terminal hydroxyl group or the hydroxyl group adjacent to the terminal hydroxyl group is oriented in the opposite direction with respect to a protective layer, and therefore adhesion of the hydroxyl groups with respect to the protective layer is less likely to be obtained. In addition, the lubricating layer containing such a compound cannot obtain sufficient hydrophobicity.

In addition, favorable corrosion resistance test results were obtained in Examples 1 to 10 in which R², R⁴, and R⁶ in Formula (1) were any one of PFPE chains selected from Formulae (5), (6), and (7) and in Examples 11 to 20 in which R², R⁴, and R⁶ were two types of PFPE chains selected from Formulae (5), (6), and (7).

In addition, the corrosion resistance test results were E in both Comparative Examples 7 and 13 in which R³ in Formula (1) was Formula (2) (in Formula (2), a was 1 and b was 1), R⁵ was Formula (3) (in Formula (3), c was 1 and d was 1), and R¹ was —OH. It is inferred that this may be because the hydrophobicity of lubricating layers is insufficient, sufficient adhesion of hydroxyl groups in R¹ with respect to protective layers is not obtained, and the coating rates of the lubricating layers are low.

### INDUSTRIAL APPLICABILITY

By using the lubricant for a magnetic recording medium containing the fluorine-containing ether compound of the present invention, a lubricating layer highly effective in suppressing corrosion of a magnetic recording medium can be formed.

### REFERENCE SIGNS LIST

10 Magnetic recording medium
11 Substrate
12 Adhesive layer
13 Soft magnetic layer
14 First underlayer
15 Second underlayer
16 Magnetic layer
17 Protective layer
18 Lubricating layer

1. A fluorine-containing ether compound represented by Formula (1) below,
R¹—CH₂—R²—CH₂—R³—CH₂—R⁴—CH₂—R⁵—CH₂—R⁶—CH₂—R⁷ (1)
(in Formula (1), R², R⁴, and R⁶ are the same or different perfluoropolyether chains, R³ is a linking group represented by Formula (2) below, R⁵ is a linking group represented by Formula (3) below, and R¹ and R⁷ are each independently a terminal group having two or three polar groups, in which individual polar groups are bound to different carbon atoms and the carbon atoms to which the polar groups are bound are bound to each other via a linking group containing a carbon atom to which no polar group is bound), and 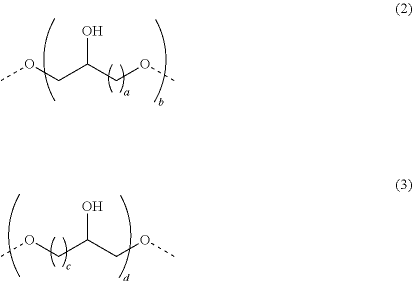
(in Formula (2), a is an integer of 1 to 3 and b is an integer of 1 to 2, and in Formula (3), c is an integer of 1 to 3 and d is an integer of 1 to 2, provided that a in Formula (2) and c in Formula (3) are not 1 at the same time).

2. The fluorine-containing ether compound according to claim 1,
wherein a in Formula (2) above is an integer of 2 to 3, and c in Formula (3) above is an integer of 2 to 3.

3. The fluorine-containing ether compound according to claim 1,
wherein all of the polar groups in R¹ and R⁷ in Formula (1) above are hydroxyl groups.

4. The fluorine-containing ether compound according to claim 1,
wherein —R¹ and —R⁷ in Formula (1) above are each independently a terminal group represented by Formulae (4-1) to (4-5) below, 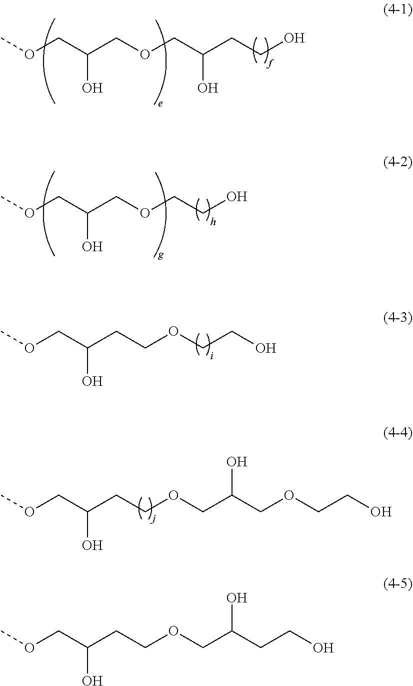
(in Formula (4-1), e is an integer of 0 to 1 and f is an integer of 1 to 4),
(in Formula (4-2), g is an integer of 1 to 2 and h is an integer of 1 to 3),
(in Formula (4-3), i is an integer of 1 to 3), and
(in Formula (4-4), j is an integer of 1 to 2).

5. The fluorine-containing ether compound according to claim 1,
wherein R², R⁴, and R⁶ in Formula (1) above are each independently any of Formulae (5) to (9) below,
—CF₂O—(CF₂CF₂O)ₖ—(CF₂O)ₗ—CF₂— (5)
(in Formula (5), k and l indicate the average degree of polymerization and each independently represent 0.1 to 20),
—CF₂O—(CF₂CF₂O)ₘ—CF₂— (6)
(in Formula (6), m indicates the average degree of polymerization and represents 0.1 to 20),
—CF₂CF₂O—(CF₂CF₂CF₂₀)ₙ—CF₂CF₂— (7)
(in Formula (7), n indicates the average degree of polymerization and represents 0.1 to 20),
—CF₂CF₂CF₂O—(CF₂CF₂CF₂CF₂₀)ₒ—CF₂CF₂CF₂— (8)
(in Formula (8), o indicates the average degree of polymerization and represents 0.1 to 10), and
—CF(CF₃)O—(CF₂CF(CF₃)O)ₚ—CF(CF₃)— (9)
(in Formula (9), p indicates the average degree of polymerization and represents 0.1 to 20).

6. The fluorine-containing ether compound according to claim 1,
wherein R¹ and R⁷ in Formula (1) above are the same as each other.

7. The fluorine-containing ether compound according to claim 1,
wherein R² and R⁶ in Formula (1) above are the same as each other.

8. The fluorine-containing ether compound according to claim 1,
wherein a number average molecular weight thereof is within a range of 500 to 10,000.

9. A lubricant for a magnetic recording medium comprising:
the fluorine-containing ether compound according to claim 1.

10. A magnetic recording medium,
wherein at least a magnetic layer, a protective layer, and a lubricating layer are sequentially provided on a substrate, and
wherein the lubricating layer contains the fluorine-containing ether compound according to claim 1.

11. The magnetic recording medium according to claim 10,
wherein an average film thickness of the lubricating layer is 0.5 nm to 2.0 nm.
